(19) United States
(12) Patent Application Publication
(10) Pub. No.: US 2006/0234986 A1
(43) Pub. Date: Oct. 19, 2006

(54) Use of TAFI inhibitors for enhanced myocardial reperfusion and facilitated PCI

(75) Inventors: Brad Buckman, Oakland, CA (US); William Dole, Ipswich, MA (US); Kohichi Kawai, Mihama-ku (JP); Michael John Morser, San Francisco, CA (US); Mariko Nagashima, Belmont, CA (US); Ronald Vergona, Sonoma, CA (US); Yi-Xin Wang, Lafayette, CA (US)

Correspondence Address: BERLEX BIOSCIENCES;PATENT DEPARTMENT, 2600 HILLTOP DRIVE, P.O. BOX 4099, RICHMOND, CA, 94804-0099, US

(73) Assignee: Schering Aktiengesellschaft, Berlin (DE)

(21) Appl. No.: 11/402,499

(22) Filed: Apr. 11, 2006

Related U.S. Application Data
(60) Provisional application No. 60/673,119, filed on Apr. 18, 2005.

(51) Int. Cl. A61K 31/445; A61K 31/195; A61K 31/66

(52) U.S. Cl. 514/114; 514/315; 514/562

(57) ABSTRACT

This invention relates to TAFI inhibitors and their use to enhance myocardial reperfusion and facilitate percutaneous coronary intervention (PCI) in the treatment of acute ST elevation myocardial infarction (STEMI).

This application claims priority to U.S. Provisional application Ser. No. 60/673,119, filed Apr. 18, 2005, the disclosure of which is incorporated herein by reference.

### FIELD OF THE INVENTION

This invention relates to the use of TAFI inhibitors to enhance myocardial reperfusion and facilitate percutaneous coronary intervention (PCI) in the treatment of acute ST elevation myocardial infarction (STEMI).

### BACKGROUND OF THE INVENTION

The fibrinolytic system removes fibrin clots from the circulation in order to maintain vessel patency. The first step in fibrinolysis is generation of a limited amount of plasmin (an active serine protease) from Glu-plasminogen by plasminogen activators such as tissue-type plasminogen activator (tPA). On the clot surface, plasmin initiates clot lysis by proteolytic cleavage of internal lysine residues in the Aα-chain of fibrin. These newly exposed C-terminal lysine and arginine residues provide further binding sites for both plasminogen and tPA, thereby amplifying plasmin production and enhancing fibrinolysis. The fibrinolytic system is regulated through inhibition of plasmin by α2-antiplasmin and inhibition of plasminogen activators by plasminogen activator inhibitor-1. In addition, plasma carboxypeptidase B, also known as TAFI (Thrombin-Activatable Fibrinolysis Inhibitor), regulates fibrinolysis. Upon activation by the thrombin/thrombomodulin complex and/or plasmin, active TAFI (TAFIa) inhibits the amplification of plasmin production by removing the newly exposed C-terminal lysine residues from partially degraded fibrin (for reviews, see Nesheim et al., 2001, Ann N Y Acad Sci 936:247-260; Bouma et al., 2001, Thromb Res 101:329-354; Schatteman et al., 2001, Clin Appl Thromb Hemost 7:93-101). Generation of TAFIa has been demonstrated during thrombus formation and subsequent thrombolytic treatment in a dog coronary artery thrombosis model (Mattsson et al., 2002, Thromb Haemost 87:557-562). Therefore, TAFI inhibitor is expected to enhance fibrinolysis by promoting plasmin production.

Evidence that TAFI inhibition enhances endogenous fibrinolysis has been reported. In a rabbit jugular vein thrombosis model, a thrombus formed in the presence of a TAFI inhibitor was lysed more readily (Minnema et al., 1998, J Clin Invest 101:10-14). In a rat model of intravascular fibrin deposition, intravenous injection of TAFIa resulted in increased pulmonary fibrin deposition in response to batroxobin challenge (Wu et al., 2003, Thromb Haemost 90:414421). In other studies, TAFI inhibition has been shown to increase endogenous fibrinolysis, resulting in decreased tissue fibrin deposition (Nerme et al., 2000, Fibrinolysis & Proteolysis 14:69; Muto et al., 2003, Eur J Pharmacol 461:181-189; Suzuki et al., 2004, J Pharmacol Exp Ther 309:607-615). These data indicate that TAFI plays a role in the regulation of endogenous fibrinolysis.

TAFI inhibition has been proven to be effective in enhancing tPA-induced fibrinolysis in several animal models using CPI, a selective peptidic TAFI inhibitor isolated from potato (Klement et al., 1999, Blood 94:2735-2743; Nagashima et al., 2000, Thromb Res 98:333-342; Refino et al., 2000, Fibrinolysis & Proteolysis 14:305-314).

In summary, available data support the therapeutic concept that TAFI inhibition, by enhancing the rate and extent of fibrinolysis, can improve reperfusion following thrombotic vascular occlusion.

The safety of TAFI inhibition has been demonstrated in TAFI-deficient mice (Nagashima et al., 2002a, J Clin Invest 109:101-110; Nagashima et al., 2002b, Front Biosci 7:d556-d568). The targeted disruption of the TAFI gene did not result in an abnormal phenotype, and TAFI-deficient mice demonstrated similar responses to various acute stimuli as their wild-type littermates. Furthermore, TAFI deficiency did not cause spontaneous bleeding or alterations in bleeding time. In the presence of low-molecular weight heparin, blood loss in TAFI-deficient mice was not significantly different from that observed in wild-type mice.

The etiology of acute myocardial infarction (AMI) in the majority of patients involves rupture or erosion of an atherosclerotic plaque with thrombotic occlusion of the coronary artery. In 1997, an estimated 1.5 million patients were discharged from U.S. hospitals diagnosed with AMI. AMI and coronary heart disease are among the leading causes of death in the U.S.

Current treatment for AMI is the expeditious restoration of normal blood flow in the infarct-related artery. This is achieved either by thrombolytic therapy or percutaneous coronary intervention (PCI) employing angioplasty with or without stent placement. In conjunction with PCI, heparin and aspirin are routinely used as antithrombotic drugs.

Currently, thrombolytic therapy remains the standard of care for patients with acute ST elevation myocardial infarction (STEMI), since PCI is not universally available. A disadvantage of PCI for STEMI is that it requires a highly trained team and a well-equipped catheterization laboratory not available in all hospitals. Furthermore, there are generally longer time delays for the initiation of PCI compared with thrombolytic therapy. As with thrombolytic therapy, the time to treatment with PCI is a critical factor that determines clinical outcomes and mortality.

PCI has several advantages over thrombolytic therapy, including:
PCI achieves both higher patency and greater blood flow in the infarct-related artery (TIMI grade 3; 90% vs<75% with thrombolysis at 90 minutes). The “Thrombolysis in Myocardial Infarction” (TIMI) grade has been widely accepted as a measure of the extent of coronary perfusion (Chesebro et al., 1987, Circulation 76:142-154) and has been used as the primary angiographic efficacy endpoint in many previous clinical trials (Anderson and Willerson, 1993, N Eng J Med 329:703-709; Lenderink et al., 1995, Circulation 92:1110-1116; Simes et al., 1995, Circulation 91:1923-1928);
Primary angioplasty is associated with reduced mortality and lower rates of bleeding, particularly intracranial hemorrhage (Li and Herrmann, 2000, Am Heart J 140:S125-S135; Zijistra, 2001, Heart 85:705-709);
The use of stents following angioplasty has also been shown to prevent reocclusion and reduce the incidence of restenosis following angioplasty in AMI; and
Compared with thrombolytic therapy, treatment of patients with PCI leads to better clinical outcomes.

The strategy of facilitated reperfusion and PCI for treatment of AMI involves initiating pharmacologic treatment before patients are transported to the cardiac catheterization laboratory for PCI. This is intended to combine the best aspects of pharmacologic thrombolysis (earliest possible reperfusion) and primary angioplasty (complete reperfusion, lower bleeding risk, relief of coronary obstruction) to further improve the clinical benefit-to-risk ratio.

Potential advantages of “facilitated PCI” include:
Enhanced TIMI flow and microvascular reperfusion (early reperfusion in more patients with pharmacologic therapy and complete and sustained reperfusion with PCI);
Reduction in the combined incidence of death, recurrent ischemia, heart failure, and need for urgent repeat revascularization;
Improved patient stability in the catheterization laboratory;
Greater technical procedural success with reduced rate of re-occlusion; and
Cost-effectiveness due to reduced need for additional interventions.

In earlier clinical trials, rescue angioplasty performed after thrombolytic therapy with full doses of tPA resulted in higher rates of mortality, reinfarction, bleeding and re-intervention. Since these early trials were conducted, there have been improvements in both interventional techniques and fibrinolytic therapy. More recent clinical studies have demonstrated that early reperfusion (spontaneous or pharmacologicaly-mediated) before PCI can significantly improve clinical outcomes (Stone et al., 2001, Circulation 104:636-641; Brodie et al., 2000, Am J Cardiol 85:13-18; and Zijistra et al., 2002, J Am Coll Cardiol 39:1733-1737).

A series of studies of primary angioplasty in AMI (PAMI trials) concluded that clinical outcomes were greatly improved in patients who underwent primary PCI and in whom TIMI-3 flow was present before angioplasty. Among 2507 patients enrolled in the four PAMI trials, spontaneous reperfusion (TIMI-3 flow) was present in 16% of patients at initial angiography (all patients received aspirin and heparin in the emergency room before angiography). The results of this study indicate that early reperfusion (TIMI-3 flow) prior to PCI is a powerful and independent predictor of in-hospital and long-term survival (Stone et al., 2001, supra). Furthermore, the Moses Cone Hospital Registry showed that when reperfusion is restored before primary angioplasty, other clinical outcomes were better with less cardiogenic shock, improved procedural success, smaller infarct size, better preservation of left ventricular function and lower 30-day mortality (Brodie et al., 2000, supra). In another study, the presence of TIMI-3 flow before angioplasty was a major determinant of clinical outcomes including better procedural success, infarct size, and 30-day mortality (Zijlstra et al., 2002, supra).

The trials described above support the therapeutic concept that pharmacologic therapy to promote early reperfusion before mechanical intervention may further improve the results of PCI. It has been reported that, using ST-segment resolution as a marker for tissue-level reperfusion, combination therapy with low-dose alteplase and GPIIb/IIIa inhibitor abciximab prior to PCI improved tissue-level reperfusion and clinical outcomes (de Lemos et al., 2001, Am Heart J 141:592-598). Recent studies with the GPIIb/IIIa antagonists or lower dose tPA have demonstrated the feasibility of testing the facilitated PCI strategy in clinical trials (PACT trial: Ross et al., 1999, J Am Coll Cardiol 34:1954-1962; ADMIRAL trial: Montalescot et al., 2001, N Engl. J Med 344:1895-1903; CADILLAC trial: Stone et al., 2002, N Engl J Med 346:957-966).

Strategies using platelet GPIIb/IIIa inhibitor abciximab and half-dose tPA enhanced early reperfusion and showed trends toward lower rates of death, reinfarction and urgent revascularization. However, this strategy increased major bleeding (SPEED trial: Speed Investigators, 2000, Circulation 101:2777-2782).

TAFI inhibitors are known in the art and include compounds such as those disclosed in WO 03/080631, WO 03/13526, WO 00/66550, WO 00/66557, WO 00/66152, WO 03/027128, WO 01/19836 and WO 02/14285. The entirety of each of these publications disclosing TAFI inhibitors is incorporated herein by reference. Known TAFI inhibitors further include AZD-9684 (Astra Zeneca) and EF-6265 (Meiji Seika Kaisha).

### SUMMARY OF THE INVENTION

The present invention involves the novel use of TAFI inhibitors to enhance myocardial reperfusion and facilitate PCI in the treatment of acute STEMI.

Accordingly, in one aspect, the invention is directed to a method of enhancing myocardial reperfusion and facilitating PCI by administering a TAFI inhibitor to a patient in need thereof.

In a presently preferred embodiment, administration of a TAFI inhibitor is made prior to PCI for acute STEMI. Treatment with a TAFI inhibitor prior to PCI is expected to enhance endogenous fibrinolysis and increase the rate and extent of myocardial reperfusion during transport of the patient to the cardiac catherization laboratory. A TAFI inhibitor has no direct effects on coagulation factors or platelet function and is not expected to increase bleeding risk.

Numerous classes of compounds that inhibit TAFI are known in the art, including compounds disclosed in WO 03/080631, WO 03/13526, WO 00/66550, WO 00/66557, WO 00/66152, WO 03/027128, WO 01/19836 and WO 02/14285; the entirety of each of these publications is incorporated herein by reference. Known TAFI inhibitors further include AZD-9684 (Astra Zeneca) and EF-6265 (Meiji Seika Kaisha). Preferred TAFI inhibitors include compounds of formulae (I), (II) and (III) disclosed in WO 03/080631 (Schering Aktiengesellschaft).

Thus, in another aspect, the invention is directed to a method of enhancing myocardial reperfusion and facilitating PCI using TAFI inhibitors of the following formula (I) or a pharmaceutically acceptable salt thereof: 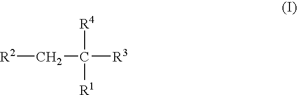
wherein:
R¹ is hydrogen, alkyl, alkenyl, aralkyl, or aralkenyl;
R² is —SH, —S—C(O)—R⁸, —P(O)(OR⁵)₂, —P(O)(OR⁵)R⁶, —P(O)(OR⁵)—R⁷—N(R⁶)₂, —P(O)(OR⁵)—R⁷—C(O)—R⁸, —P(O)(OR⁵)—R⁷—N(R⁵)—C(O)OR⁸, —P(O)(OR⁵)—R⁷—N(R⁵)—C(O)—R⁷—N(R⁵)—C(O)OR⁸, —P(O)(OR⁵)—R⁷—N(R⁵)—S(O)₂—R⁹, or —P(O)(OR⁵)—R⁷—N(R⁵)—C(S)—N(R⁶)₂;
R³ is tetrazole, —C(O)OR⁶, —C(O)O—R⁷—OC(O)R⁵, —S(O)OR⁵, —S(O)₂OR⁵, —P(O)(OR⁵)₂, —P(O)(OR⁵)R⁶, or —B(OR⁵)₂;
R⁴ is aryl optionally substituted by one or more substituents selected from the group consisting of alkyl, halo, haloalkyl, haloalkoxy, mercapto, alkylthio, phenyl, cycloalkyl, nitro, cyano, —OR⁶, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently cycloalkylene (optionally substituted by alkyl), a straight or branched alkylene chain (optionally substituted by hydroxy, mercapto, alkylthio, aryl, cycloalkyl, —N(R⁶)₂, —C(O)OR⁶, or —C(O)N(R⁶)₂), or a straight or branched alkenylene chain (optionally substituted by hydroxy, mercapto, alkylthio, aryl, cycloalkyl, —N(R⁶)₂, —C(O)OR⁶, or —C(O)N(R⁶)₂);
each R⁸ is independently alkyl, alkenyl, aryl, aralkyl or aralkenyl; and
R⁹ is —R⁷N(R⁶)C(O)OR⁸, haloalkyl, alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), or N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶);
provided that when R³ is —C(O)OH or when R⁴ is a substituted aryl or substituted N-heterocyclyl, R² can not be —P(O)(OR⁵)—R⁷—N(H)—C(O)OR⁸ or —P(O)(OR⁵)—R⁷—N(H)—C(O)—R⁷—N(R⁵)—C(O)OR⁸;
as a single stereoisomer, a mixture of stereoisomers, or as a racemic mixture of stereoisomers.

In another aspect, the invention is directed to a method of enhancing myocardial reperfusion and facilitating PCI using TAFI inhibitors of the following formula (II) or a pharmaceutically acceptable salt thereof: 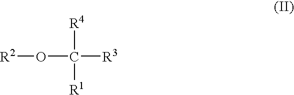
wherein:
R¹ is hydrogen, alkyl, alkenyl, aryl or aralkenyl;
R² is —P(O)(OR⁵)₂, —P(O)(OR⁵)R⁶, —P(O)(OR⁵)—R⁷—N(R⁶)₂, —P(O)(OR⁵)—R⁷—C(O)—R⁸, —P(O)(OR⁵)—R⁷—N(R⁵)—C(O)OR⁸, —P(O)(OR⁵)—R⁷—N(R⁵)—C(O)—R⁷—N(R⁵)-C(O)OR”, —P(O)(OR⁵)—R⁷—N(R⁵)—S(O)₂—R⁹, or —P(O)(OR⁵)—R⁷—N(R⁵)—C(S)—N(R⁶)₂;
R³ is tetrazole, —C(O)OR⁶, —C(O)O—R⁷—OC(O)R⁵, —S(O)OR⁵, S(O)₂OR⁵, —P(O)(OR⁵)₂, —P(O)(OR⁵)R⁶, or —B(OR⁵)₂;
R⁴ is aryl optionally substituted by one or more substituents selected from the group consisting of alkyl, halo, haloalkyl, haloalkoxy, mercapto, alkylthio, phenyl, cycloalkyl, nitro, cyano, —OR⁶, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the

N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently cycloalkylene (optionally substituted by alkyl), a straight or branched alkylene chain (optionally substituted by hydroxy, mercapto, alkylthio, aryl, cycloalkyl, —N(R⁶)₂, —C(O)OR⁶, or —C(O)N(R⁶)₂), or a straight or branched alkenylene chain (optionally substituted by hydroxy, mercapto, alkylthio, aryl, cycloalkyl, —N(R⁶)₂, —C(O)OR⁶, or —C(O)N(R⁶)₂);
each R⁸ is independently alkyl, alkenyl, aryl, aralkyl or aralkenyl; and
R⁹ is —R⁷N(R⁶)C(O)OR⁸, haloalkyl, alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), or N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶);
provided that when R³ is —C(O)OH or when R⁴ is a substituted aryl or substituted N-heterocyclyl, R² can not be —P(O)(OR⁵)—R⁷—N(H)—C(O)OR⁸ or —P(O)(OR⁵)—R⁷—N(H)—C(O)—R⁷—N(R⁵)—C(O)OR⁸;
as a single stereoisomer, a mixture of stereoisomers, or as a racemic mixture of stereoisomers.

In another aspect, the invention is directed to a method of enhancing myocardial reperfusion and facilitating PCI using TAFI inhibitors of formula (III) or a pharmaceutically acceptable salt thereof: 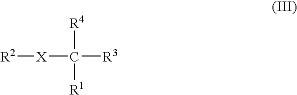
wherein:
X is —CH₂— or —O—;
R¹ is hydrogen, alkyl, alkenyl, aryl or aralkenyl;
R² is —P(O)(OR⁵)—R⁷—N(R⁵)—C(O)R⁶, —P(O)(OR⁵)—R⁷—N(R⁵)—C(O)OR⁸ or —P(O)(OR⁵)—R⁷—N(R⁵)—C(O)—R⁷—N(R⁵)-C(O)OR⁸,
R³ is —C(O)OH;
R⁴ is aryl optionally substituted by one or more substituents selected from the group consisting of alkyl, halo, haloalkyl, haloalkoxy, mercapto, alkylthio, phenyl, cycloalkyl, nitro, cyano, —OR⁵, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR ⁵)—N(R⁵)₂, —N(R⁵)—C(O)—N(R⁶)₂ or —N(R⁵)—C(O)—R⁷—N(R⁶₂), or wherein a nitrogen atom in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently cycloalkylene (optionally substituted by alkyl), a straight or branched alkylene chain (optionally substituted by hydroxy, mercapto, alkylthio, aryl, cycloalkyl, —N(R⁶)₂, —C(O)OR⁶, or —C(O)N(R⁶)₂), or a straight or branched alkenylene chain (optionally substituted by hydroxy, mercapto, alkylthio, aryl, cycloalkyl, —N(R⁶)₂, —C(O)OR⁶, or —C(O)N(R⁶)₂); and
each R³ is independently alkyl, alkenyl, aryl, aralkyl or aralkenyl;
as a single stereoisomer, a mixture of stereoisomers, or as a racemic mixture of stereoisomers.

In another aspect, the invention is directed to a method of enhancing myocardial reperfusion and facilitating PCI using TAFI inhibitors that are known in the art.

Known TAFI inhibitors include compounds of the following formula (IV), which are disclosed in WO 00/66550 (AstraZeneca), the entirety of which is incorporated herein by reference: 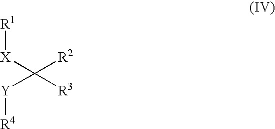
wherein the following substituents are (as defined in WO 00/66550):
R¹ represents C₁-C₆ alkyl, substituted with one or more basic groups such as amino, amidino and/or guanidino; cycloalkyl, substituted with one or more basic groups such as amino, amidino and/or guanidino; heterocyclyl, containing at least one nitrogen atom; heterocyclyl, containing at least one hetero atom selected from S or 0, and substituted with one or more basic groups such as amino, amidino and/or guanidino; or aryl, substituted with one or more basic groups such as amino, amidino and/or guanidino;
R² represents H, acyl, acylamino, alkyl, alkyl, alkylcarbamoyl, althylthio, alkoxy, aroyl, aroylamino, aryloxy, arylthio, amidino, amino, aryl, carbamoyl, carboxy, cyano, cycloalkyl, formyl, guanidino, halogen, heterocyclyl, hydroxy, oxo, nitro, thiol, Z₂N—CO—O, ZO—CO—NZ— or Z₂N—CO—NZ— group;
R³ represents COOR⁵, SO(OR⁵), SO₃R⁵, P═O(OR⁵)₂, B(OR⁵)₂, P═OR⁵(OR⁵), or tetrazole, or any carboxylic acid isostere;
R⁴ represents a 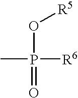
-group, a 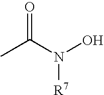
-group, or a 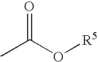
-group;
R⁵ represents H, C₁-C₆ alkyl or aryl;
R⁶ represents C₁-C₆ alkyl, aryl, cycloalkyl, heterocyclyl, or an optionally N-substituted H₂N—C(Z)—CONH—C(Z)— or H₂N—C(Z)— group;
R⁷ represents H or C₁-C₆ alkyl;
X represents O, S, SO, SO₂, C(Z)₂, N(Z), NR⁷SO₂, SO₂NR⁷, NR⁷CO or CONR⁷;
Y represents O, N(Z), S, C(Z)₂, or a single bond; and
Z represents independently H, C₁-C₆ alkyl, aryl, cycloalkyl or heterocyclyl, with the proviso that when X represents O, S, SO, SO₂, N(Z), NR⁷SO₂, SO₂NR⁷ or NR⁷CO then Y represents C(Z)₂, or a single bond;
or a pharmaceutically acceptable salt or solvate thereof, or a solvate of such a salt.

Known TAFI inhibitors include compounds of the following formula (V), which are disclosed in WO 00/66557 (AstraZeneca), the entirety of which is incorporated herein by reference: 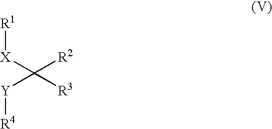
wherein the following substituents are (as defined in WO 00/66557):
R¹ represents C₁-C₆ alkyl, substituted with one or more basic groups such as amino, amidino and/or guanidino; cycloalkyl, substituted with one or more basic groups such as amino, amidino and/or guanidino; heterocyclyl, containing at least one nitrogen atom; heterocyclyl, containing at least one hetero atom selected from S or O, and substituted with one or more basic groups such as amino, amidino and/or guanidino; or aryl, substituted with one or more basic groups such as amino, amidino and/or guanidino;
R² represents H, acyl, acylamino, alkyl, alkyl, alkylcarbamoyl, althylthio, alkoxy, aroyl, aroylamino, aryloxy, arylthio, amidino, amino, aryl, carbamoyl, carboxy, cyano, cycloalkyl, formyl, guanidino, halogen, heterocyclyl, hydroxy, oxo, nitro, thiol, Z₂N—CO—O, ZO—CO—NZ— or Z₂N—CO—NZ— group;
R³ represents COOR⁵, SO(OR⁵), SO₃R⁵, P═O(OR⁵)₂, B(OR⁵)₂, P═OR⁵(OR⁵), or tetrazole, or any carboxylic acid isostere;
R⁴ represents SH, S—CO—C₁-C₆ alkyl, or S—CO-aryl;
R⁵ represents H, C₁-C₆ alkyl or aryl;
R⁶ represents H or C₁-C₆ alkyl;
X represents O, S, SO, SO₂, C(Z)₂, N(Z), NR⁶SO₂, SO₂NR⁶, NR⁶CO or CONR⁶;
Y represents C(Z)₂; and
Z represents independently H, C₁-C₆ alkyl, aryl, cycloalkyl or heterocyclyl;
or a pharmaceutically acceptable salt or solvate thereof, or a solvate of such a salt.

Known TAFI inhibitors include compounds of the following formula (VI), which are disclosed in WO 03/027128 (AstraZeneca), the entirety of which is incorporated herein by reference: 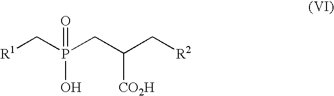
wherein the following substituents are (as defined in WO 03/027128):
R¹ is phenyl (optionally substituted by halogen, nitro, cyano, hydroxy, C₁-C₆ alkyl (itself optionally substituted by halogen, hydroxy or S(O)₂R³, C₁-C₆ alkoxy (itself optionally substituted by halogen), C₁-C₆ alkylthio (itself optionally substituted by halogen), phenyl, phenylcarbonyl, phenyloxy, heteroaryl, S(O)₂R⁴ or S(O)₂NHR⁵, wherein the foregoing phenyl and heteroaryl rings are optionally substituted by halogen, hydroxy, C₁-C₆ alkyl (itself optionally substituted by halogen) or C₁-C₆ alkoxy (itself optionally substituted by halogen)), 9,10-dihydroanthracenyl (optionally substituted by oxo), naphthyl (optionally substituted by halogen, C₁-C₆ alkyl (itself optionally substituted by halogen) or C₁-C₆ alkoxy (itself optionally substituted by halogen)) or heteroaryl (optionally substituted by halogen, C₁-C₆ alkyl (itself optionally substituted by halogen), C₁-C₆ alkoxy (itself optionally substituted by halogen) or phenylcarbonyl (itself optionally substituted by halogen));
R² is aminopyridinyl, aminothiazolyl or 3-azabicyclo[3.2.1]octyl;
R³ is C₁-C₆ alkyl or phenyl;
R⁴ is C₁-C₆ alkyl;
R⁵ is (CH₂)₂R⁶; and
R⁶ is phenyl or heteroaryl, either of which is optionally substituted by halogen, C₁-C₆ alkyl (itself optionally substituted by halogen) or C₁-C₆ alkoxy (itself optionally substituted by halogen);
or a pharmaceutically acceptable salt or solvate thereof, or a solvate of such a salt.

Known TAFI inhibitors further include 3-mercapto-propionic acid derivative compounds of the following formula, disclosed in Polla et al., 2004, Bioorg Med Chem 12:1151-1175, the entirety of which is incorporated herein by reference: 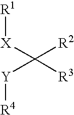
wherein
R¹ represents pyridinyl, substituted with one or more groups selected from the group consisting of amino, methyl, chloro and ethyl;
R² represents H, hydroxyl, fluoro, methyl or ethyl;
R³represents COOH;
R⁴ represents SH; and
X and Y represent CH₂.

Known TAFI inhibitors include compounds of the following formula (VII), which are disclosed in WO 03/013526 (Merck), the entirety of which is incorporated herein by reference: 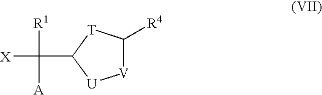
wherein the following substituents are (as defined in WO 03/013526):
T is or N(R^{2″}), U is C(R³) or N(R^{2′}), and V is C(R²), N or N(R²), provided that when T is N and U is C(R³), then V is N(R²), when T is N and U is N(R^{2′}), then V is C(R²), and when T is N(R²) and U is C(R³), then V is N or N(R²);
A is COOR⁵, tetrazole, or a carboxylic acid isotere, wherein R⁵ is hydrogen, an unsubstituted C₁-C₈ alkyl, wherein the alkyl substituent is selected from the group consisting of aryl, heterocycle, NR⁶R⁷, OR⁶, and CHR⁶OC(O)R⁷, wherein R⁶ and R⁷ are independently selected from the group consisting of hydrogen, C₁-C₆ alkyl, and aryl;
X is C₁-C₆ alkyl, substituted with one or more basic groups, or Y—W, wherein Y is (CR⁸R⁹), (CR⁸R⁹)(CR¹⁰R¹¹), (CR⁸R⁹)(CR¹⁰R¹¹)(CR¹²R¹³), or a bond, wherein R⁸, R¹⁰, and R¹² are independently selected from the group consisting of hydrogen, C₁-C₄ alkyl, OR¹⁴, F and NR¹⁴R¹⁵, wherein R¹⁴ and R¹⁵ are independently selected from the group consisting of hydrogen and C₁-C₄ alkyl, and wherein R⁹, R¹¹, and R¹³ are independently selected from the group consisting of hydrogen, F and C₁-C₄ alkyl, and wherein W is a C₃-C₇ cycloalkyl ring wherein at least one ring carbon atom is substituted with a basic group, a 4- to 7-membered saturated or unsaturated heterocyclic ring, having 1-4 nitrogen ring atoms, wherein each ring carbon atom is independently unsubstituted or mono- or bi-substituted with a basic group, halogen, or C₁-C₄ alkyl, or a 6- to 10-membered aryl ring system, wherein at least one ring carbon atom is substituted with a basic group;
R¹ is selected from the group consisting of hydrogen, C₁-C₄ alkyl, OR¹⁶, F, and NR¹⁶R¹⁷, wherein R¹⁶ and R¹⁷ are independently selected from the group consisting of hydrogen and C₁-C₄ alkyl;
R² is selected from the group consisting of hydrogen, methyl, phenyl, unsubstituted or independently mono or bi-substituted with a substituent selected from the group selected from the group consisting of halogen, phenyl, C₁-C₄ alkyl, CF₃, CN, OCH₃, NH₂, NO₂, pyridine and pyrimidine, C₁-C₄ alkenyl, A¹-(A²)₀₋₁-(A³)₀₋₁-(A⁴)₀₋₁-A⁵, wherein
   A¹ is C₁-C₇ alkylene, wherein each carbon atom is independently unsubstituted or mono- or di-substituted with a substituent selected from the group consisting of F, CF₃ and C₁-C₄ alkyl;
   A² is selected from the group consisting of C(O), C(O)NH, NHC(O), and —NHSO₂;
A³ is a bond or C₁-C₃ alkylene, where each carbon atom is independently unsubstituted or mono- or di-substituted with C₁-C₄ alkyl;
   A⁴ is a bond, O, or OCH₂; and
   A⁵ is phenyl, unsubstituted or independently mono- or di-substituted with a substituent selected from the group consisting of halogen, phenyl, C₁-C₄ alkyl, CF₃, CN, OCH₃ and NH₂, pyridinyl, naphthyl, CF₃, C₁-C₅ alkyl, —NR¹⁸R¹⁹, wherein R¹⁸ and R¹⁹ are independently selected from the group consisting of hydrogen and C₁-C₄ alkyl, OH, COOH, C₃-C₁₀ carbocyclic ring system, unsubstituted or independently mono- or di-substituted with a substituent selected from the group consisting of NH₂ and C₁-C₄ alkyl, 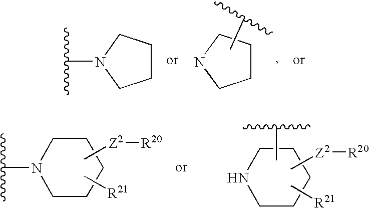
      wherein Z² is a bond or C₁-C₄ alkylene, R²⁰ and R²¹ are independently selected from the group consisting of hydrogen, phenyl, CN or difluorophenyl;
R^{2′} is selected from the group consisting of hydrogen, methyl, phenyl, unsubstituted or independently mono- or di-substituted with a substituent selected from the group consisting of halogen, phenyl, C₁-C₄ alkyl, CF₃, CN, OCH₃, NH₂, NO₂, pyridine and pyrimidine, C₁-C₄ alkenyl, and A^{1′}-(A^{2′})₀₋₁-(A^{3′})₀₋₁-(A^{4′})₀₋₁-A^{5′}, wherein
   A^{1′} is C₁-C₇ alkylene, wherein each carbon atom is independently unsubstituted or mono- or di-substituted with a substituent selected from the group consisting of F, CF₃ and C₁-C₄ alkyl;
   A^{2′} is selected from the group consisting of C(O), C(O)NH, NHC(O), and —NHSO₂;
   A^{3′} is a bond or C₁-C₃ alkylene, where each carbon atom is independently unsubstituted or mono- or di-substituted with C₁-C₄ alkyl;
   A^{4′} is a bond, O, or OCH₂; and
   A^{5′} is phenyl, unsubstituted or independently mono- or di-substituted with a substituent selected from the group consisting of halogen, phenyl, C₁-C₄ alkyl, CF₃, CN, OCH₃ and NH₂, pyridinyl, naphthyl, CF₃, C₁-C₅ alkyl, —NR^{18′}R^{19′}, wherein R^{18′} and R^{19′} are independently selected from the group consisting of hydrogen and C₁-C₄ alkyl, OH, COOH, C₃-C₂₀ carbocyclic ring system, unsubstituted or independently mono- or di-substituted with a substituent selected from the group consisting of NH₂ and C₁-C₄ alkyl, 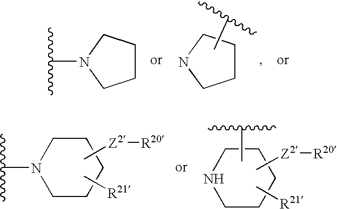
      wherein Z^{2′} is a bond or C₁-C₄ alkylene, R^{20′} and R^{21′} are independently selected from the group consisting of hydrogen, phenyl, CN or difluorophenyl;
R^{2″} is selected from the group consisting of hydrogen, methyl, phenyl, unsubstituted or independently mono- or di-substituted with a substituent selected from the group consisting of halogen, phenyl, C₁-C₄ alkyl, CF₃, CN, OCH₃, NH₂, NO₂, pyridine and pyrimidine, C₁-C₄ alkenyl, and A^{1″}-(A^{2″})₀₋₁-(A^{3″})₀₋₁-(A^{4″})₀₋₁-A^{5″}, wherein
   A^{1″} is C₁-C₇ alkylene, wherein each carbon atom is independently unsubstituted or mono- or di-substituted with a substituent selected from the group consisting of F, CF₃ and C₁-C₄ alkyl;
   A^{2″} is selected from the group consisting of C(O), C(O)NH, NHC(O), and —NHSO₂;
   A^{3″} is a bond or C₁-C₃ alkylene, where each carbon atom is independently unsubstituted or mono- or di-substituted with C₁-C₄ alkyl;
   A^{4″} is a bond, O, or OCH₂; and
   A^{5″}is phenyl, unsubstituted or independently mono- or di-substituted with a substituent selected from the group consisting of halogen, phenyl, C₁-C₄ alkyl, CF₃, CN, OCH₃ and NH₂, pyridinyl, naphthyl, CF₃, C₁-C₅ alkyl, —NR^{18′″}R^{19″}, wherein R^{18″} and R^{19″} are independently selected from the group consisting of hydrogen and C₁-C₄ alkyl, OH, COOH, C₃-C₁₀ carbocyclic ring system, unsubstituted or independently mono- or di-substituted with a substituent selected from the group consisting of NH₂ and C₁-C₄ alkyl, 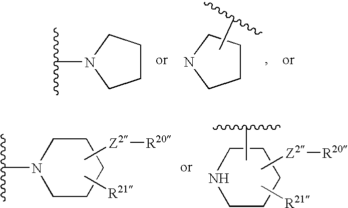
      wherein Z^{2″} is a bond or C₁-C₄ alkylene, R^{20″} and R^{21″} are independently selected from the group consisting of hydrogen, phenyl, CN or difluorophenyl;
R³ is hydrogen, unsubstituted or substituted C₁-C₆ alkyl, unsubstituted or substituted phenyl, unsubstituted or substituted naphthyl, or unsubstituted or substituted heterocycle,
   wherein one or more substituents in substituted alkyl is independently selected from the group consisting of F, C₁-C₆ alkyl, phenyl, naphthyl, and heterocycle, and one or more substituents in substituted phenyl, substituted naphthyl and substituted heterocycle is independently selected from the group consisting of phenyl, naphthyl, heterocycle, —CF₃, —CN, C₁-C₆ alkyl, hydroxy, C₁-C₆ alkoxy, halogen, —NO₂, —NR²³R²⁴, —SO₂R²³, —SO₂NR²³R²⁴, —CONR²³R²⁴, or —COR²³, wherein R²³ and R²⁴ are independently selected hydrogen and C₁-C₄ alkyl; and
R⁴ is hydrogen, unsubstituted or substituted C₁-C₆ alkyl, unsubstituted or substituted phenyl, unsubstituted or substituted naphthyl, unsubstituted or substituted heterocycle, or unsubstituted or substituted C₁-C₄ alkylenearyl,
   wherein one or more substituents in substituted alkyl is independently selected from the group consisting of F, C₁-C₆ alkyl, phenyl, naphthyl, and heterocycle, and one or more substituents in substituted phenyl, substituted naphthyl and substituted heterocycle is independently selected from the group consisting of phenyl, naphthyl, heterocycle, —CF₃, —CN, C₁-C₆ alkyl, hydroxy, C₁-C₆ alkoxy, halogen, —NO₂, —NR²⁵R²⁶, —SO₂R²⁵, —SO₂NR²⁵R²⁶, —CONR²⁵R²⁶, or —COR²⁵, wherein R²⁵ and R²⁶ are independently selected hydrogen and C₁-C₄ alkyl;
or a pharmaceutically acceptable salt thereof.

Known TAFI inhibitors include compounds of the following formula (VIII), which are disclosed in WO 01/19836 and US 2003/0119787 (Meiji Seika Kaisha), the entireties of which are incorporated herein by reference: 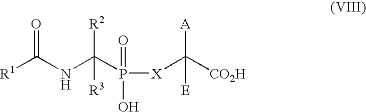
wherein the following substituents are (as defined in WO 01/19836):
R¹ represents hydrogen atom, an alkyl group, a substituted alkyl group, an alkoxy group, a substituted alkoxy group, amino group, a substituted amino group, a carboxylic group, a substituted carboxylic group, a heterocyclic group, or a substituted heterocyclic group;
R² and R³ may be the same or different and independently represent hydrogen atom, an alkyl group, a substituted alkyl group, an alkoxy group, a substituted alkoxy group, amino group, a substituted amino group, a carboxylic group, a substituted carboxylic group, a heterocyclic group, or a substituted heterocyclic group, or R² and R³ may form a 5 to 7 membered carbon-ring together with the carbon atom to which they bind;
X represents —CH2—, —O—, or —NH—;
A represents a group of the following formula (i): 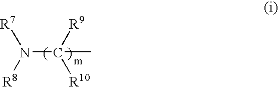
   [in which R⁷ and R⁸ may be the same or different and independently represent hydrogen atom, a halogen atom, hydroxyl group, phenyl group, an alkyl group, an alkoxyl group, an aryloxy group, an acyl group, carboxyl group, an alkoxycarbonyl group, carbamoyl group, a substituted carbamoyl group, amino group, a substituted amino group, nitro group, —SR¹¹ group (R¹¹ represents an alkyl group, phenyl group, or a substituted phenyl group), SOR¹² group (R¹² represents an alkyl group, phenyl group, a substituted phenyl group, an alkoxyl group, an aryloxy group, amino group, or a substituted amino group), or SO₂R¹³ group (R¹³ represents an alkyl group, phenyl group, a substituted phenyl group, an alkoxyl group, an aryloxy group, amino group, or a substituted amino group);
   m represents an integer of from 1 to 8; and —(C)ₘ— represents a saturated or unsaturated carbon chain], or
A represents a group of the following formula (ii): 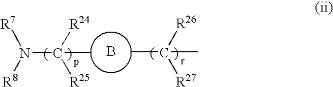
   [in which R⁷ and R⁸ may be the same or different and independently represent hydrogen atom, an alkyl group, an acyl group, an alkoxycarbonyl group, an aryloxycarbonyl group, carbamoyl group, a substituted carbamoyl group, or amidino group; R²⁴, R²⁵, R²⁶ and R²⁷ may be the same or different and independently represent hydrogen atom, a halogen atom, hydroxyl group, an alkyl group, amino group, or a substituted amino group; p and q independently represent an integer of from 0 to 2; —(C)ₚ— and —(C)_{q} independently represent single bond, or a saturated or unsaturated carbon chain; and B represents a carbocyclic group, a substituted carbocyclic group; a heterocyclic group, or a substituted heterocyclic group], or
A represents a group of the following formula (iii): 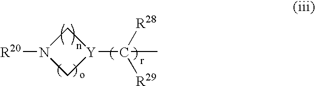
   [in which R²⁰ represents hydrogen atom, an alkyl group, an acyl group, an alkoxycarbonyl group, carbamoyl group, an alkylcarbamoyl group, or amidino group; R²⁸ and R²⁹ may be the same or different and independently represent hydrogen atom, a halogen atom, hydroxyl group, an alkyl group, amino group, or an alkylamino group; n and o independently represent an integer of from 0 to 5; r represents an integer of from 0 to 4; —(C)ᵣ— independently represents single bond, or a saturated or unsaturated carbon chain;
   and Y represents CH or nitrogen atom], or
A represents a group of the following formula (iv): 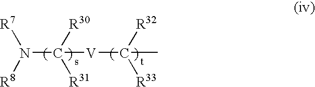
   [in which R⁷ and R⁸ may be the same or different and independently represent hydrogen atom, an alkyl group, an acyl group, an alkoxycarbonyl group, an aryloxycarbonyl group, carbamoyl group, a substituted carbamoyl group, or amidino group; R³⁰, R³¹, R³² and R³³ may be the same or different and independently represent hydrogen atom, a halogen atom, hydroxyl group, an alkyl group, oxo group, amino group, or alkylamino group; s represents an integer of from 0 to 3; t represents an integer of from 1 to 3; —(C)ₛ— and —(C)ₜ— independently represent single bond, or a saturated or unsaturated carbon chain;
   and V represents —O— or —NH—]; and,
   E represents hydrogen atom, or CH2CH2- to form together with a piperidine ring group or a N-substituted piperidine ring group;
or a pharmacologically acceptable salt thereof, or a hydrate thereof or a solvate thereof.

Known TAFI inhibitors further include the following compound EF6265 (Meiji Seika Kaisha), which is disclosed in Suzuki et al., 2004, supra and U.S. Pat. No. 6,576,627, the entireties of which are incorporated herein by reference.

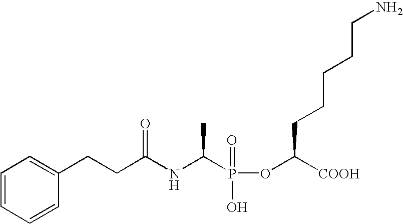

Known TAFI inhibitors include compounds of the following formula (IX), which are disclosed in WO 02/14285 (Pfizer), the entirety of which is incorporated herein by reference: 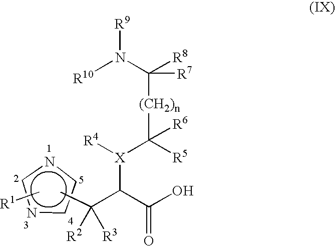
wherein the following substituents are (as defined in WO 02/14285):
X is N or CH;
N is 0 to 3
R¹ is (a) C₁-C₆ alkyl, straight chain or branched chain, (b) C₁-C₆ alkenyl, straight chain or branched chain, (c) C₁-C₆ alkynyl, straight chain or branched chain, (d) heterocycle, (e) aromatic heterocycle, (f) aryl, (g) hydrogen, said groups (a), (b) and (c) optionally further substituted by C₃-C₇ cycloalkyl, aryl, aromatic heterocycle, heterocycle, OR¹¹, NR¹¹R¹², S(O)ₚNR¹¹, OC(O)R¹¹, CO₂R¹¹, CONR¹¹R¹², SO₂NR¹¹R¹², halo and NHSO₂R¹¹, where R¹may be attached at any position on the imidazole ring;
R² and R³ are each independently selected from hydrogen, C₁-C₆ alkyl, optionally further substituted by OR¹¹, halo, or wherein R and R³ may be joined to form a link, said link is C₂-C₆ alkylene;
R⁴is hydrogen, C₁-C₆ alkyl, optionally further substituted by C₃-C₇ cycloalkyl, aryl, OR¹¹, halo and R¹¹, or wherein R⁴ and R¹⁰ may be joined to form a link, wherein said link is C₁-C₄ alkylene, optionally further substituted by OR¹¹, halo and R¹¹;
R⁵ and R⁶are selected from: hydrogen, aryl, C₁-C₆ alkyl, said alkyl optionally further substituted by C₃-C₇ cycloalkyl, aromatic heterocycle, heterocycle, aryl, OR¹¹, R¹¹ and halo, or wherein R¹⁰ and either of R⁵ or R⁶ may be joined to form a link, wherein said link is C₁-C₃ alkylene, optionally further substituted by OR¹¹, halo R¹¹ and aryl, or wherein R⁵ or R⁶ may be joined to form a link, wherein said link is C₂-C₆ alkylene;
R⁷ and R⁸ are independently selected from: hydrogen, C₁-C₆ alkyl, optionally further substituted by OR¹¹, halo, aryl and R¹¹, or wherein R⁷ and R⁸ may be joined to form a link, wherein said link is C₂-C₆ alkylene;
R⁹ and R¹⁰ are independently selected from: hydrogen, C(NR¹¹)NR¹¹R¹², C₁-C₆ alkyl, said alkyl optionally further substituted by OR¹¹, halo, aryl and R¹¹, or wherein R⁹ and R¹⁰ may be joined to form a link, wherein said link is C₂-C₆ alkylene;
R¹¹ and R¹² are each independently selected from hydrogen or C₁-C₆ alkyl, or when forming a NR¹¹R¹², moiety, R¹¹ and R¹² may also be joined to form a link wherein said link is C₂-C₆ alkylene;
p is 0, 1 or 2;
wherein
Aryl is defined as a 6-14 membered aromatic carbocycle, optionally further substituted by R¹¹, halo, OR¹¹, NR¹¹R¹², NR¹¹CO₂R¹², C0₂R¹¹, NR¹¹SO₂R¹², CN, haloalkyl, O(haloalkyl), SO₂NR¹¹R¹², C(O)NR¹¹R¹²;
Aromatic heterocycle is defined as a 5 to 7 membered ring, containing from 1 to 3 heteroatoms, each independently selected from O, S and N, said heterocycle group optionally substituted by OR¹¹, NR¹¹R¹², CO₂R¹¹, NR¹¹CO₂R¹², R¹¹, halo, CN, haloalkyl, O(haloalkyl), S(O)ₚR¹¹, OC(O)R¹¹, NR¹¹SO₂R¹², SO₂NR¹¹R¹², C(O)NR¹¹R¹²; and
Heterocycle is defined as a 3-8 membered ring containing from 1-3 heteroatoms, each independently selected from O, S and N, said ring being saturated or partially saturated, said heterocycle group optionally substituted by OR¹¹, NR¹¹R¹², CO₂R¹¹, NR¹¹CO₂R¹², R¹¹, halo, CN, haloalkyl, O(haloalkyl), S(O)ₚR¹¹, OC(O)R¹¹, NR¹¹SO₂R¹², SO₂NR¹¹R¹², C(O)NR¹¹R¹²;
or a pharmaceutically acceptable salt, solvate or prodrug thereof.

### DETAILED DESCRIPTION OF THE INVENTION

### A. DEFINITIONS

As used in the specification and appended claims, unless specified to the contrary, the following terms have the meaning indicated:

The terms “a” and “an” mean one or more.

“Alkyl” refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl(iso-propyl), n-butyl, n-pentyl, 1,1-dimethylethyl(t-butyl), and the like. Unless stated otherwise specifically in the specification, the alkyl radical may be optionally substituted by hydroxy, alkoxy, aryloxy, haloalkoxy, cyano, nitro, mercapto, alkylthio, cycloalkyl, —N(R⁶)₂, —C(O)OR⁶, —C(O)N(R⁶)₂ or —N(R⁶)—C(O)—R⁶ where each R⁶ is as defined in the Summary of the Invention. Unless stated otherwise specifically in the specification, it is understood that for radicals (as defined below) that contain a substituted alkyl group, the substitution can occur on any carbon of the alkyl group.

“Alkoxy” refers to a radical of the formula —ORₐ where Rₐ is an alkyl radical as defined above, e.g., methoxy, ethoxy, n-propoxy, 1-methylethoxy(isopropoxy), n-butoxy, n-pentoxy, 1,1-dimethylethoxy(t-butoxy), and the like. Unless stated otherwise specifically in the specification, it is understood that for radicals (as defined below) that contain a substituted alkoxy group, the substitution can occur on any carbon of the alkoxy group. The alkyl radical in the alkoxy radical may be optionally substituted as described above.

“Alkylthio” refers to a radical of the formula —SRₐ where Rₐ is an alkyl radical as defined above, e.g., methylthio, ethylthio, n-propylthio, 1-methylethylthio(isopropylthio), n-butylthio, n-pentylthio, 1,1-dimethylethylthio(t-butylthio), and the like. Unless stated otherwise specifically in the specification, it is understood that for radicals (as defined below) that contain a substituted alkylthio group, the substitution can occur on any carbon of the alkylthio group. The alkyl radical in the alkylthio radical may be optionally substituted as described above.

“Alkenyl” refers to a straight or branched hydrocarbon chain radical consisting solely of carbon and hydrogen atoms, containing at least one double bond, having from two to eight carbon atoms, and which is attached to the rest of the molecule by a single bond or a double bond, e.g., ethenyl, prop-1-enyl, but-1-enyl, pent-1-enyl, penta-1,4-dienyl, and the like. Unless stated otherwise specifically in the specification, the alkenyl radical may be optionally substituted by hydroxy, alkoxy, haloalkoxy, cyano, nitro, mercapto, alkylthio, cycloalkyl, —N(R⁶)₂, —C(O)OR⁶, —C(O)N(R⁶)₂ or —N(R⁶)—C(O)—R⁶ where each R⁶ is as defined in the Summary of the Invention. Unless stated otherwise specifically in the specification, it is understood that for radicals (as defined below) that contain a substituted alkenyl group, the substitution can occur on any carbon of the alkenyl group.

“Alkynyl” refers to a straight or branched monovalent hydrocarbon chain radical consisting solely of carbon and hydrogen atoms, containing at least one triple bond, having from two to eight carbon atoms, and which is attached to the rest of the molecule by a single bond, e.g., ethynyl, prop-1-ynyl, but-1-ynyl, pent-1-ynyl, pent-3-ynyl, and the like. Unless stated otherwise specifically in the specification, the alkynyl radical may be optionally substituted by hydroxy, alkoxy, haloalkoxy, cyano, nitro, mercapto, alkylthio, cycloalkyl, —N(R⁶)₂, —C(O)OR⁶, —C(O)N(R⁶)₂ or —N(R⁶)—C(O)—R⁶ where each R⁶ is as defined in the Summary of the Invention. Unless stated otherwise specifically in the specification, it is understood that for radicals (as defined below) that contain a substituted alkynyl group, the substitution can occur on any carbon of the alkynyl group.

“Aryl” refers to a phenyl or naphthyl radical. Unless stated otherwise specifically in the specification, the term “aryl” or the prefix “ar-” (such as in “aralkyl”) is meant to include aryl radicals optionally substituted by one or more substituents selected from the group consisting of alkyl, halo, nitro, cyano, haloalkyl, haloalkoxy, mercapto, alkylthio, phenyl, cycloalkyl, —OR⁶ (including hydroxy and alkoxy), —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)OR⁶, —R⁷—C(O)OR⁶, —C(O)—N(R⁶)₂, —R⁷—C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂) where each R⁵, R⁶, R⁷, and R⁸ are as defined above in the Summary of the Invention.

“Aralkyl” refers to a radical of the formula —R^{a}R^{b} where Rₐ is an alkyl radical as defined above and R_{b} is one or more aryl radicals as defined above, e.g., benzyl, diphenylmethyl and the like. The aryl radical(s) may be optionally substituted as described above.

“Aralkoxy” refers to a radical of the formula —OR_{d} where R_{d} is an aralkyl radical as defined above, e.g., benzyloxy, and the like. The aryl radical may be optionally substituted as described above.

“Aralkenyl” refers to a radical of the formula —R_{c}R_{b} where R_{c} is an alkenyl radical as defined above and R_{b} is one or more aryl radicals as defined above, e.g., 3-phenylprop-1-enyl, and the like. The aryl radical(s) and the alkenyl radical may be optionally substituted as described above.

“Alkylene chain” refers to a straight or branched divalent hydrocarbon chain consisting solely of carbon and hydrogen, containing no unsaturation and having from one to eight carbon atoms, e.g., methylene, ethylene, propylene, n-butylene, and the like. The alkylene chain may be optionally substituted by one or more substituents selected from the group consisting of aryl, halo, hydroxy, alkoxy, haloalkoxy, cyano, nitro, mercapto, alkylthio, cycloalkyl, —N(R⁶)₂, —C(O)OR⁶, —C(O)N(R⁶)₂ or —N(R⁶)—C(O)—R⁶ where each R⁶ is as described above in the Summary of the Invention. The alkylene chain may be attached to the rest of the molecule through any two carbons within the chain.

“Alkenylene chain” refers to a straight or branched divalent hydrocarbon chain consisting solely of carbon and hydrogen, containing at least one double bond and having from two to eight carbon atoms, e.g., ethenylene, prop-1-enylene, but-1-enylene, pent-1-enylene, hexa-1,4-dienylene, and the like. The alkenylene chain may be optionally substituted by one or more substituents selected from the group consisting of aryl, halo, hydroxy, alkoxy, haloalkoxy, cyano, nitro, mercapto, alkylthio, cycloalkyl, —N(R⁶)₂, —C(O)OR⁶, —C(O)N(R⁶)₂ or —N(R⁶)—C(O)—R⁶ where each R⁶ is as described above in the Summary of the Invention. The alkenylene chain may be attached to the rest of the molecule through any two carbons within the chain.

“Cycloalkyl” refers to a stable monovalent monocyclic or bicyclic hydrocarbon radical consisting solely of carbon and hydrogen atoms, having from three to ten carbon atoms, and which is saturated and attached to the rest of the molecule by a single bond, e.g., cyclopropyl, cyclobutyl, cyclopentyl, cyclohexyl, decalinyl and the like. Unless otherwise stated specifically in the specification, the term “cycloalkyl” is meant to include cycloalkyl radicals which are optionally substituted by one or more substituents independently selected from the group consisting of alkyl, aryl, aralkyl, halo, haloalkyl, hydroxy, alkoxy, haloalkoxy, cyano, nitro, mercapto, alkylthio, cycloalkyl, —N(R⁶)₂, —C(O)OR⁶, —C(O)N(R⁶)₂ or —N(R⁶)—C(O)—R⁶ where each R⁶ is as defined in the Summary of the Invention.

“Cycloalkylene” refers to a stable divalent monocyclic or bicyclic hydrocarbon consisting solely of carbon and hydrogen atoms, having from three to ten carbon atoms, and which is saturated and attached to the rest of the molecule by two single bonds, e.g., cyclopropylene, cyclobutylene, cyclopentylene, cyclohexylene, decalinylene and the like. Unless otherwise stated specifically in the specification, the term “cycloalkylene” is meant to include cycloalkylene moieties which are optionally substituted by one or more substituents independently selected from the group consisting of alkyl, aryl, aralkyl, halo, haloalkyl, hydroxy, alkoxy, haloalkoxy, cyano, nitro, mercapto, alkylthio, cycloalkyl, —N(R⁶)₂, —C(O)OR⁶, —C(O)N(R⁶)₂ or —N(R⁶)—C(O)—R⁶ where each R⁶ is as defined in the Summary of the Invention.

“N-heterocyclyl” refers to a stable 3- to 15-membered ring radical which consists of carbon atoms and from one to five heteroatoms selected from the group consisting of nitrogen, oxygen and sulfur, wherein at least one of the heteroatoms is a nitrogen. For purposes of this invention, the N-heterocyclyl radical may be a monocyclic, bicyclic or tricyclic ring system, which may include fused or bridged ring systems; and the nitrogen, carbon or sulfur atoms in the N-heterocyclyl radical may be optionally oxidized; the nitrogen atom may be optionally quaternized; and the N-heterocyclyl radical may be partially or fully saturated or aromatic. The N-heterocyclyl radical may be attached to the main structure at any heteroatom or carbon atom that results in the creation of a stable compound. Examples of such N-heterocyclyl radicals include, but are not limited to, azepinyl, azetidinyl, benzimidazolyl, benzoxazolyl, carbazolyl, decahydroisoquinolyl, quinuclidinyl, imidazolyl, imidazolinyl, imidazolidinyl, isothiazolidinyl, indolyl, isoindolyl, indolinyl, isoindolinyl, indolizinyl, isoxazolyl, isoxazolidinyl, morpholinyl, benzothiadiazolyl, oxadiazolyl, octahydroindolyl, octahydroisoindolyl, 2-oxopiperazinyl, 2-oxopiperidinyl, 2-oxopyrrolidinyl, 2-oxoazepinyl, oxazolyl, oxazolidinyl, perhydroazepinyl, piperidinyl, piperazinyl, 4-piperidonyl, phenazinyl, phenothiazinyl, phenoxazinyl, phthalazinyl, pteridinyl, purinyl, pyrrolyl, pyrrolidinyl, pyrazolyl, pyrazolidinyl, pyridinyl, pyrazinyl, pyrimidinyl, pyridazinyl, quinazolinyl, quinoxalinyl, quinolinyl, quinuclidinyl, isoquinolinyl, thiazolyl, thiazolidinyl, thiadiazolyl, triazolyl, tetrazolyl, tetrahydroisoquinolyl, thiomorpholinyl, thiomorpholinyl sulfoxide, and thiomorpholinyl sulfone. The carbon atoms in the N-heterocyclyl radical may be optionally substituted by alkyl, halo, nitro, cyano, haloalkyl, haloalkoxy, mercapto, alkylthio, phenyl, cycloalkyl, —OR⁶, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)OR⁶, —R⁷—C(O)OR⁶, —C(O)—N(R⁶)₂, —R⁷—C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂) where each R⁵, R⁶, R⁷ and R⁸ are as defined above in the Summary of the Invention. The nitrogen atoms in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂ where each R⁵, R⁶ and R⁷ are as defined above in the Summary of the Invention. Preferred N-heterocyclyl radicals are piperidinyl, tetrahydrosoquinolinyl, or benzothiadiazolyl.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like.

“Haloalkoxy” refers to a radical of the formula —OR_{c} where R_{c} is an haloalkyl radical as defined above, e.g., trifluoromethoxy, difluoromethoxy, trichloromethoxy, 2,2,2-trifluoroethoxy, 1-fluoromethyl-2-fluoroethoxy, 3-bromo-2-fluoropropoxy, 1-bromomethyl-2-bromoethoxy, and the like.

“Mammal” includes humans and domesticated animals, such as cats, dogs, swine, cattle, sheep, goats, horses, rabbits, and the like.

“Optional” or “optionally” means that the subsequently described event or circumstance may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

“TAFI” refers to Thrombin Activatable Fibrinolysis Inhibitor, also known as plasma procarboxypeptidase B, which when activated gives rise to an active basic carboxypeptidase called activated TAFI or TAFIa. TAFIa is also known as carboxypeptidase U or carboxypeptidase R.

“Therapeutically effective amount” refers to that amount of a compound of the invention which, when administered to a human in need thereof, is sufficient to effect the enhancement of myocardial reperfusion and the facilitation of PCI, and especially when used in the treatment of acute STEMI. The amount of a compound of the invention which constitutes a “therapeutically effective amount” will vary depending on the compound, the condition and its severity, the age of the human to be treated, and the like, but can be determined routinely by one of ordinary skill in the art having regard to his own knowledge and to this disclosure.

“Treating” or “treatment” as used herein covers the treatment of a disease-state in a mammal, preferably a human, which disease-state is characterized by thrombotic activity, and includes:

(i) preventing the condition from occurring in a human, in particular, when such human is predisposed to the condition but has not yet been diagnosed as having it;

(ii) inhibiting the condition, i.e., arresting its development; or

(iii) relieving the condition, i.e., causing regression of the condition.

The compounds of the invention, or their pharmaceutically acceptable salts may contain one or more asymmetric centers and may thus give rise to enantiomers, diastereomers, and other stereoisomeric forms that may be defined, in terms of absolute stereochemistry, as (R)— or (S)— or, as (D)- or (L)- for amino acids. The present invention is meant to include all such possible isomers, as well as, their racemic and optically pure forms. Optically active (+) and (−), (R)— and (S)—, or (D)- and (L)- isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

The nomenclature used herein is a modified form of the I.U.P.A.C. nomenclature system wherein the compounds of the invention are named herein as derivatives of the acid moiety. For example, the following compound of formula (III) wherein R¹ is hydrogen, R² is —P(O)(OH)—R⁷—N(H)—C(O)OR⁸ (where R⁷ is hexyl and R⁸ is benzyl), R³ is —C(O)OH, and R⁴ is 3-guanidinophenyl, i.e., the compound of the following formula: 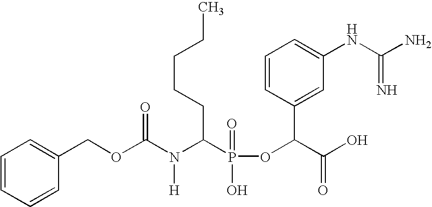
is named herein as 2-(3-guanidinophenyl)-2-((1-(benzyloxycarbonyl)aminohexyl)-(hydroxy)phosphinoyl)oxyethanoic acid. Unless otherwise indicated, compound names are intended to include any single stereoisomer, enantiomer, diastereomer, racemate or mixture of stereoisomers.

The use of parentheses in a formula herein is used to conserve space. Accordingly, the use of parenthesis in a formula indicates that the group enclosed within the parentheses is attached directly to the atom preceding the parenthesis. For example, the term —P(O)(OR⁵)—R⁷—N(R⁵)—C(O)—R⁷—N(R⁵)—C(O)OR⁸ can be drawn as follows:

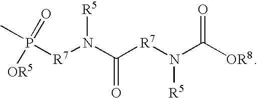

### B. UTILITY OF THE INVENTION

The compounds of the invention are inhibitors of TAFI. According to the present invention, the compounds are useful in enhancing myocardial reperfusion and in facilitating percutaneous coronary intervention (PCI). Administration of a TAFI inhibitor is expected to further enhance endogenous fibrinolysis, thereby increasing the percentage of patients with spontaneous reperfusion prior to PCI. It is expected that a TAFI inhibitor will enhance fibrinolysis without affecting coagulation or platelet function.

The compounds of the invention may also be combined and/or coadministered with other therapeutic agents such as, but not limited to, antithrombotics (including antiplatelet agents, anticoagulants and profibrinolytics), antihypertensives, agents to treat dyslipidaemia (e.g., statins such as LIPITOR™), Factor Xa inhibitors, and antiarrhythmics (e.g., amiodarone and digoxin). Suitable antithrombotics include aspirin, clopidogrel, ticlopidine, warfarin, unfractionated heparin, hirudin, streptokinase, urokinase, recombinant tissue-type plasminogen activator (tPA), tenecteplase (TNKase), DSPA, dipyridamole, REOPRO™, AGGRASTAT™, and INTEGRILIN™. A TAFI inhibitor is not expected to further increase bleeding risk when given together with anti-platelet or anticoagulant drugs.

### C. ADMINISTRATION OF THE TAFI INHIBITORS

Administration of the TAFI inhibitors, or their pharmaceutically acceptable salts, in pure form or in an appropriate pharmaceutical composition, for use in treatment according to the present invention, can be carried out via any of the accepted modes of administration of agents for serving similar utilities. The pharmaceutical compositions of the present invention may be in any form that allows for the composition to be administered to a patient. Typical routes of administration include, without limitation, oral, topical, transdermal, inhalation, parenteral, sublingual, rectal, vaginal, and intranasal. The term “parenteral” as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the invention, or a pharmaceutically acceptable salt thereof, for treatment of a disease-state characterized by thrombotic activity, i.e., by the formation of a thrombus, or by hypercoagulability, in accordance with the teachings of this invention.

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, e.g., inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, e.g., a gelatin capsule, it may contain, in addition to materials of the above type, a liquid carrier such as polyethylene glycol or a fatty oil.

The pharmaceutical composition may be in the form of a liquid, e.g., an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the invention intended for either parenteral or oral administration should contain an amount of a compound of the invention such that a suitable dosage will be obtained. Typically, this amount is at least 0.01% of a compound of the invention in the composition. When intended for oral administration, this amount may be varied to be between 0.1 and about 70% of the weight of the composition. Preferred oral pharmaceutical compositions contain between about 4% and about 50% of the compound of the invention. Preferred pharmaceutical compositions and preparations according to the present invention are prepared so that a parenteral dosage unit contains between 0.01 to 1% by weight of the compound of the invention.

The pharmaceutical composition of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the invention from about 0.1 to about 10% w/v (weight per unit volume).

The pharmaceutical composition of the invention may be intended for rectal administration, in the form, e.g., of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the invention in solid or liquid form may include an agent that binds to the compound of the invention and thereby assists in the delivery of the compound. Suitable agents that may act in this capacity include a monoclonal or polyclonal antibody, a protein or a liposome.

The pharmaceutical composition of the invention may consist of dosage units that can be administered as an aerosol. The term “aerosol” is used to denote a variety of systems ranging from those of colloidal nature to systems consisting of pressurized packages. Delivery may be by a liquefied or compressed gas or by a suitable pump system that dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

Whether in solid, liquid or gaseous form, the pharmaceutical composition of the present invention may contain one or more known pharmacological agents used in the treatment of disease-states characterized by thrombotic activity.

The pharmaceutical compositions of the invention may be prepared by methodology well known in the pharmaceutical art. For example, a pharmaceutical composition intended to be administered by injection can be prepared by combining a compound of the invention with water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

The compounds of the invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disease-state; and the host undergoing therapy. Generally, a therapeutically effective daily dose is from about 0.14 mg to about 14.3 mg/kg of body weight per day of a compound of the invention, or a pharmaceutically acceptable salt thereof; preferably, from about 0.7 mg to about 10 mg/kg of body weight per day; and most preferably, from about 1.4 mg to about 7.2 mg/kg of body weight per day. For example, for administration to a 70 kg person, the dosage range would be from about 10 mg to about 1.0 gram per day of a compound of the invention, or a pharmaceutically acceptable salt thereof, preferably from about 50 mg to about 700 mg per day, and most preferably from about 100 mg to about 500 mg per day.

### D. PREFERRED EMBODIMENTS

Of the compounds of the invention as set forth above in the Summary of the Invention, several groups of compounds are particularly preferred.

Of the compounds of formula (I) as set forth above in the Summary of the Invention, a preferred group is that group of compounds of formula (I) wherein:
R¹ is hydrogen;
R²is —SH or —S—C(O)—R⁸;
R³ is tetrazole, —C(O)OR⁶ or —C(O)O—R⁷—OC(O)R⁵;
R⁴is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶²), or wherein a nitrogen atom in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain optionally substituted by hydroxy, mercapto, alkylthio, aryl, cycloalkyl, —N(R⁶)₂, —C(O)OR⁶, or—C(O)N(R⁶)₂; and
each R⁸ is independently alkyl, alkenyl, aryl, aralkyl or aralkenyl.

Of this preferred group of compounds, a preferred subgroup is that subgroup of compounds of formula (I) wherein:
R¹ is hydrogen;
R² is —SH or —S—C(O)—R⁸;
R³ is —C(O)OR⁶;
R⁴ is aryl optionally substituted by one or more substituents selected from the group consisting of halo, nitro, —N(R⁶)₂, —R⁷—N(R⁶)₂ and —N(R⁵)—C(NR⁵)—N(R⁵)₂;
each R⁵ is independently hydrogen, alkyl or aralkyl;
each R⁶ is independently hydrogen, alkyl, aryl or aralkyl;
R⁷ is a straight or branched alkylene chain; and
R⁸ is independently alkyl, alkenyl, aryl, aralkyl or aralkenyl.

Of this preferred subgroup of compounds of formula (I), preferred compounds are selected from the group consisting of the following:
2-(4-guanidinophenyl)-3-mercaptopropanoic acid;
2-(3-guanidinophenyl)-3-mercaptopropanoic acid;
2-(3-aminophenyl)-3-mercaptopropanoic acid; and
2-(2-chloro-5-guanidinophenyl)-3-mercaptopropanoic acid.

Of the preferred group of compounds of formula (I) as set forth above, another preferred subgroup is that subgroup of compounds of formula (I) wherein:
R¹ is hydrogen;
R² is —SH, or —S—C(O)—R⁸;
R³ is —C(O)OR⁶;
R⁴ is 3(4)-piperidinyl wherein the nitrogen atom in the piperidinyl radical is optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, aryl or aralkyl;
R⁷ is a straight or branched alkylene chain optionally substituted by hydroxy, mercapto, alkylthio, aryl, cycloalkyl, —N(R⁶)₂, —C(O)OR⁶, or —C(O)N(R⁶)₂; and
R⁸ is alkyl, alkenyl, aryl, aralkyl or aralkenyl.

Of this preferred subgroup of compounds of formula (I), preferred compounds are selected from the group consisting of the following:
2-(piperidin-4-yl)-3-mercaptopropanoic acid;
2-(1-amidinopiperidin-4-yl)-3-mercaptopropanoic acid;
2-(1-(1-iminoethyl)piperidin-4-yl)-3-mercaptopropanoic acid;
2-(1-(aminomethylcarbonyl)piperidin-4-yl)-3-mercaptopropanoic acid;
2-(piperidin-3-yl)-3-mercaptopropanoic acid; and
2-(1-amidinopiperidin-3-yl)-3-mercaptopropanoic acid.

Of the compounds of formula (I) as set forth above in the Summary of the Invention, another preferred group is that group of compounds of formula (I) wherein:
R¹ is hydrogen;
R² is —P(O)(OR⁵)₂, —P(O)(OR⁵)R⁶ or —P(O)(OR⁵)—R⁷—C(O)—R⁸;
R³ is tetrazole, —C(O)OR⁶, or —C(O)O—R⁷—OC(O)R⁵;
R⁴is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷ —N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain optionally substituted by hydroxy, mercapto, alkylthio, aryl, cycloalkyl, —N(R⁶)₂, —C(O)OR⁶, or —C(O)N(R⁶)_{2;} and
each R⁸ is independently alkyl, alkenyl, aryl, aralkyl or aralkenyl.

Of this preferred group of compounds, a preferred subgroup is that subgroup of compounds of formula (I) wherein:
R¹ is hydrogen;
R² is —P(O)(OR⁵)₂, —P(O)(OR⁵)R⁶ or —P(O)(OR⁵)—R⁷—C(O)—R⁸;
R³is —C(O)OR⁶;
R⁴is aryl optionally substituted by one or more substituents selected from the group consisting of halo, nitro, —N(R⁶)₂, —R⁷—N(R⁶)₂ and —N(R⁵)—C(NR⁵)—N(R⁵)₂;
each R⁵ is independently hydrogen, alkyl or aralkyl;
each R⁶ is independently hydrogen, alkyl, aryl or aralkyl;
each R⁷ is independently a straight or branched alkylene chain optionally substituted by aryl, —N(R⁶)₂ or —C(O)OR⁶; and
R⁸ is alkyl, alkenyl, aryl, aralkyl or aralkenyl.

Of this preferred subgroup of compounds of formula (I), preferred compounds are selected from the group consisting of the following:
2-(3-guanidinophenyl)-3-phosphonopropanoic acid;
2-(3-aminophenyl)-3-((phenyl)(hydroxy)phosphinoyl)propanoic acid;
2-(3-aminophenyl)-3-((4-phenylbutyl)(hydroxy)phosphinoyl)propanoic acid;
2-(3-aminophenyl)-3-((pentyl)(hydroxy)phosphinoyl)propanoic acid;
2-(3-guanidinophenyl)-3-((phenyl)(hydroxy)phosphinoyl)propanoic acid;
2-(3-guanidinophenyl)-3-((4-phenylbutyl)(hydroxy)phosphinoyl)propanoic acid;
2-(3-guanidinophenyl)-3-((pentyl)(hydroxy)phosphinoyl)propanoic acid;
2-(3-guanidinophenyl)-3-((4-methylpentyl)(hydroxy)phosphinoyl)propanoic acid;
2-(3-guanidinophenyl)-3-((3-phenylpropyl)(hydroxy)phosphinoyl)propanoic acid;
2-(3-guanidinophenyl)-3-((3-phenylprop-2-enyl)(hydroxy)phosphinoyl)propanoic acid;
2-(3-guanidinophenyl)-3-((phenylmethyl)(hydroxy)phosphinoyl)propanoic acid;
2-(3-guanidinophenyl)-3-((pentyl)(hydroxy)phosphinoyl)propanoic acid methyl ester;
2-(3-guanidinophenyl)-3-((ethyl)(hydroxy)phosphinoyl)propanoic acid;
2-(3-guanidinophenyl)-3-((2-phenylethyl)(hydroxy)phosphinoyl)propanoic acid; and
2-(3-guanidinophenyl)-3-((2-(methylcarbonyl)ethyl)(hydroxy)phosphinoyl)propanoic acid.

Of the compounds of formula (I) as set forth above in the Summary of the Invention, another preferred group is that group of compounds of formula (I) wherein:
R¹ is hydrogen;
R² is —P(O)(OR⁵)—R⁷—N(R⁵)—C(O)OR⁸;
R³ is —C(O)OR⁶ (where R⁶ is alkyl, aryl or aralkyl);
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(N R⁵)—N(R⁵)₂, —N(R⁵)—C(O)—N(R⁶)₂ and —N(R⁵)—C(O)—R⁷—N(R⁶₂) where each R⁶ is independently hydrogen, alkyl, aryl or aralkyl;
each R⁵ is independently hydrogen, alkyl or aralkyl;
each R⁷ is a straight or branched alkylene chain optionally substituted by aryl, —N(R⁶)₂ or —C(O)OR⁶; and
each R⁸ is independently alkyl, alkenyl, aryl, aralkyl or aralkenyl.

Of this group of compounds of formula (I), preferred compounds are selected from the group consisting of the following:
2-(3-(t-butoxycarbonylamino)methylphenyl)-3-((1-(benzyloxycarbonyl)amino-2-methylpropyl)(hydroxy)phosphinoyl)propanoic acid t-butyl ester; and
2-(3-(t-butoxycarbonylamino)methylphenyl)-3-((1-(benzyloxycarbonyl)amino-2-methylpropyl)(ethoxy)phosphinoyl)propanoic acid t-butyl ester.

Of the compounds of formula (I) as set forth above in the Summary of the Invention, another preferred group is that group of compounds of formula (I) wherein:
R¹ is hydrogen;
R² is —P(O)(OR⁵)—R⁷—N(R⁶)₂ or —P(O)(OR⁵)—R⁷—N(R⁵)—C(S)—N(R⁶)₂;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
each R⁵ is independently hydrogen, alkyl or aralkyl;
each R is independently hydrogen, alkyl, alkenyl, alkynyl, aryl, aralkyl or aralkenyl;
each R⁷ is a straight or branched alkylene chain optionally substituted by hydroxy, mercapto, alkylthio, aryl, cycloalkyl, —N(R⁶)₂, —C(O)OR⁶, or —C(O)N(R⁶)₂; and
each R⁸ is independently alkyl, alkenyl, aryl, aralkyl or aralkenyl.

Of this group of compounds of formula (I), preferred compounds are selected from the group consisting of the following:
2-(3-(t-butoxycarbonylamino)methylphenyl)-3-((1-amino-2-methylpropyl)(hydroxy)-phosphinoyl)propanoic acid t-butyl ester; and
2-(3-(t-butoxycarbonylamino)methylphenyl)-3-((1-amino-2-methylpropyl)(ethoxy)-phosphinoyl)propanoic acid t-butyl ester.

Of the compounds of formula (I) as set forth above in the Summary of the Invention, another preferred group is that group of compounds of formula (I) wherein:
R¹ is hydrogen;
R² is —P(O)(OR⁵)—R⁷—N(R⁵)—S(O)₂—R⁹;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the

N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently cycloalkylene (optionally substituted by alkyl), a straight or branched alkylene chain (optionally substituted by hydroxy, mercapto, alkylthio, aryl, cycloalkyl, —N(R⁶)₂, —C(O)OR⁶, or —C(O)N(R⁶)₂), or a straight or branched alkenylene chain (optionally substituted by hydroxy, mercapto, alkylthio, aryl, cycloalkyl, —N(R⁶)₂, —C(O)OR⁶, or —C(O)N(R⁶)₂);
each R⁸ is independently alkyl, alkenyl, aryl, aralkyl or aralkenyl; and
R⁹ is —R⁷N(R⁶)C(O)OR⁸, haloalkyl, alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), or N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

Of this group of compounds, a preferred subgroup is that subgroup of compounds of formula (I) wherein:
R¹ is hydrogen;
R² is —P(O)(OR⁵)—R⁷—N(R⁵)—S(O)₂—R⁹;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸; —R⁷—N(R⁶)—C(O)OR⁸, and —N(R⁵)—C(NR⁵)—N(R⁵)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain (optionally substituted by hydroxy, mercapto, alkylthio, aryl, cycloalkyl, —N(R⁶)₂, —C(O)OR⁶, or—C(O)N(R⁶)₂),
each R⁸ is independently alkyl, alkenyl, aryl, aralkyl or aralkenyl; and
R⁹ is —R⁷N(R⁶)C(O)OR⁸, haloalkyl, alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), or N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

Of this preferred subgroup of compounds, a preferred class is that class of compounds of formula (I) wherein:
R¹ is hydrogen;
R² is —P(O)(OR⁵)—R⁷—N(R⁵)—S(O)₂—R⁹;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸; —R⁷—N(R⁶)—C(O)OR⁸, and —N(R⁵)—C(NR⁵)—N(R⁵)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain optionally substituted by hydroxy, mercapto, alkylthio, aryl, cycloalkyl, —N(R⁶)₂, —C(O)OR⁶, or—C(O)N(R⁶)₂,
each R⁸ is independently alkyl, alkenyl, aryl, aralkyl or aralkenyl; and
R⁹ is alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

Of this preferred class of compounds of formula (I), preferred compounds are selected from the group consisting of the following:
2-(3-(amino)methylphenyl)-3-((1-(3-phenylpropylsulfonyl)amino-2-methylpropyl)-(hydroxy)phosphinoyl)propanoic acid, methyl ester;
2-(3-(t-butoxycarbonylamino)methylphenyl)-3-((1-(3-phenylpropylsulfonyl)amino-2-methylpropyl)(hydroxy)phosphinoyl)propanoic acid;
2-(3-(t-butoxycarbonylamino)methylphenyl)-3-((1-(3-phenylpropylsulfonyl)amino-2-methylpropyl)(hydroxy)phosphinoyl)propanoic acid, methyl ester;
2-(3-(amino)methylphenyl)-3-((1-(3-phenylpropylsulfonyl)amino-2-methylpropyl)(hydroxy)-phosphinoyl)propanoic acid;
2R)-2-(3-(amino)methylphenyl)-3-(((1R)-1-(3-phenylpropylsulfonyl)amino-2-methylpropyl)(hydroxy)phosphinoyl)propanoic acid;
(2S)-2-(3-(amino)methylphenyl)-3-(((1R)-1-(3-phenylpropylsulfonyl)amino-2-methylpropyl)(hydroxy)phosphinoyl)propanoic acid;
(2R/S)-2-(3-(amino)methylphenyl)-3-(((1S)-1-(3-phenylpropylsulfonyl)amino-2-methylpropyl)(hydroxy)phosphinoyl)propanoic acid;
(2R/S)-2-(3-(amino)methylphenyl)-3-(((1R)-1-(3-phenylpropylsulfonyl)amino-2-methylpropyl)(hydroxy)phosphinoyl)propanoic acid;
(2R)-2-(3-(amino)methylphenyl)-3-(((1S)-1-(3-phenylpropylsulfonyl)amino-2-methylpropyl)(hydroxy)phosphinoyl)propanoic acid;
(2S)-2-(3-(amino)methylphenyl)-3-(((1S)-1-(3-phenylpropylsulfonyl)amino-2-methylpropyl)(hydroxy)phosphinoyl)propanoic acid;
2-(3-(t-butoxycarbonylamino)methylphenyl)-3-((1-(3-phenylpropylsulfonyl)amino-2-methylpropyl)(ethoxy)phosphinoyl)propanoic acid, t-butyl ester;
2-(3-(amino)methylphenyl)-3-((1-(2-phenylethylsulfonyl)amino-2-methylpropyl)-(hydroxy)phosphinoyl)propanoic acid;
2-(3-(amino)methylphenyl)-3-((1-(benzylsulfonyl)amino-2-methylpropyl)-(hydroxy)phosphinoyl)propanoic acid;
2-(3-(amino)methylphenyl)-3-((1-(2-(naphth-1-yl)ethylsulfonyl)amino-2-methylpropyl)(hydroxy)phosphinoyl)propanoic acid;
2-(3-(amino)methylphenyl)-3-((1-(3-(4-methoxyphenyl)propylsulfonyl)amino-2-methylpropyl)(hydroxy)phosphinoyl)propanoic acid;
2-(3-(amino)methylphenyl)-3-((1-(2-(4-methoxyphenyl)ethylsulfonyl)amino-2-methylpropyl)(hydroxy)phosphinoyl)propanoic acid;
2-(3-(amino)methylphenyl)-3-((1-(methylsulfonyl)amino-2-methylpropyl)(hydroxy)phosphinoyl)propanoic acid;
2-(3-(amino)methylphenyl)-3-((1-(2-benzyloxyethylsulfonyl)amino-2-methylpropyl)-(hydroxy)phosphinoyl)propanoic acid;
2-(3-(amino)methylphenyl)-3-((1-(2-hydroxyethylsulfonyl)amino-2-methylpropyl)-(hydroxy)phosphinoyl)propanoic acid;
2-(3-aminophenyl)-3-((1-(3-phenylpropylsulfonyl)amino-2-methylpropyl)-(hydroxy)phosphinoyl)propanoic acid;
2-(3-guanidinophenyl)-3-((1-(3-phenylpropylsulfonyl)amino-2-methylpropyl)(hydroxy)-phosphinoyl)propanoic acid;
2-(3-(amino)methylphenyl)-3-((1-(4-phenylbutylsulfonyl)amino-2-methylpropyl)-(hydroxy)phosphinoyl)propanoic acid, and
2-(3-(amino)methylphenyl)-3-((l-(2-phenylethenylsulfonyl)amino-2-methylpropyl)-(hydroxy)phosphinoyl)propanoic acid.

Of the preferred subgroup of compounds as set forth above, another preferred class is that class of compounds of formula (I) wherein:
R¹ is hydrogen;
R² is —P(O)(OR⁵)—R⁷—N(R⁵)—S(O)₂—R⁹;
R³ is tetrazole, —C(O)OR⁶ , or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸; —R⁷—N(R⁶)—C(O)OR⁸, and —N(R⁵)—C(NR⁵)—N(R⁵)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain optionally substituted by hydroxy, mercapto, alkylthio, aryl, cycloalkyl, —N(R⁶)₂, —C(O)OR⁶, or —C(O)N(R⁶)₂,
each R⁸ is independently alkyl, alkenyl, aryl, aralkyl or aralkenyl; and
R⁹ is aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

Of this class of compounds of formula (I), preferred compounds are selected from the group consisting of the following:
2-(3-(amino)methylphenyl)-3-((1-(naphth-1-ylsulfonyl)amino-2-methylpropyl)(hydroxy)-phosphinoyl)propanoic acid;
2-(3-(amino)methylphenyl)-3-((1-(3-trifluoromethylphenylsulfonyl)amino-2-methyl-propyl)(hydroxy)phosphinoyl)propanoic acid;
2-(3-(amino)methylphenyl)-3-((1-(4-pentylphenylsulfonyl)amino-2-methylpropyl)-(hydroxy)phosphinoyl)propanoic acid;
2-(3-(amino)methylphenyl)-3-((1-(4-acetamidophenylsulfonyl)amino-2-methylpropyl)-(hydroxy)phosphinoyl)propanoic acid;
2-(3-(amino)methylphenyl)-3-((1-(4-phenylphenylsulfonyl)amino-2-methylpropyl)-(hydroxy)phosphinoyl)propanoic acid; and
2-(3-(amino)methylphenyl)-3-((1-(phenylsulfonyl)amino-2-methylpropyl)(hydroxy)-phosphinoyl)propanoic acid.

Of the preferred subgroup of compounds as set forth above, another preferred class is that class of compounds of formula (I) wherein:
R¹ is hydrogen;
R² is —P(O)(OR⁵)—R⁷—N(R⁵)—S(O)₂—R⁹;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸; —R⁷—N(R⁶)—C(O)OR⁸, —N(R⁶)—C(O)—R⁶, —R⁷—N(R⁶)—C(O)—R⁶, and —N(R⁵)—C(NR⁵)—N(R⁵)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain (optionally substituted by hydroxy, mercapto, alkylthio, aryl, cycloalkyl, —N(R⁶)₂, —C(O)OR⁶, or—C(O)N(R⁶)₂),
each R⁸ is independently alkyl, alkenyl, aryl, aralkyl or aralkenyl; and
R⁹ is —R⁷—N(R⁶)—C(O)OR⁸.

Of this class of compounds of formula (I), a preferred compound is 2-(3-(amino)methylphenyl)-3-((1-(3-phenyl-2-(benzyloxycarbonyl)aminopropyl-sulfonyl)amino-2-methylpropyl)(hydroxy)phosphinoyl)propanoic acid.

Of the preferred subgroup of compounds as set forth above, another preferred class is that class of compounds of formula (I) wherein:
R¹ is hydrogen;
R² is —P(O)(OR⁵)—R⁷—N(R⁵)—S(O)₂—R⁹;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸; —R⁷—N(R⁶)—C(O)OR⁸, —N(R⁶)—C(O)—R⁶, —R⁷—N(R⁶)—C(O)—R⁶, and —N(R⁵)—C(NR⁵)—N(R⁵)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain (optionally substituted by hydroxy, mercapto, alkylthio, aryl, cycloalkyl, —N(R⁶)₂, —C(O)OR⁶, or —C(O)N(R⁶)₂),
each R⁸ is independently alkyl, alkenyl, aryl, aralkyl or aralkenyl; and
R⁹ is N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

Of this class of compounds of formula (I), preferred compounds are selected from the group consisting of the following:
2-(3-(amino)methylphenyl)-3-((1-(thien-2-ylsulfonyl)amino-2-methylpropyl)-(hydroxy)phosphinoyl)propanoic acid; and
2-(3-(amino)methylphenyl)-3-((1-(benzothiadiazolylsulfonyl)amino-2-methylpropyl)-(hydroxy)phosphinoyl)propanoic acid.

Of the compounds of formula (I) as set forth above in the Summary of the Invention, another preferred group is that group of compounds of formula (I) wherein:
R¹ is hydrogen;
R² is —P(O)(OR⁵)—R⁷—N(R⁵)—C(O)OR⁸;
R³ is —C(O)OR⁶;
R⁴ is unsubstituted phenyl or unsubstituted N-heterocyclyl;
each R⁵ is independently hydrogen, alkyl or aralkyl;
each R⁶ is independently hydrogen, alkyl, alkenyl, alkynyl, aryl, aralkyl or aralkenyl;
each R⁷ is a straight or branched alkylene chain optionally substituted by aryl, —N(R⁶)₂ or —C(O)OR⁶; and
R⁸ is alkyl, alkenyl, aryl, aralkyl or aralkenyl.

Of this group of compounds of formula (I), preferred compounds are selected from the group consisting of the following:
2-phenyl-3-((1-(benzyloxycarbonyl)amino-2-methylpropyl)(hydroxy)phosphinoyl)-propanoic acid; 2-tetrahydroisoquinolinyl-3-((1-(benzyloxycarbonyl)amino-2-methylpropyl)-(hydroxy)phosphinoyl)propanoic acid.

Of the compounds of formula (II) as set forth above in the Summary of the Invention, a preferred group is that group of compounds of formula (II) wherein:
R¹ is hydrogen;
R² is —P(O)(OR⁵)R⁶, —P(O)(OR⁵)—R⁷—N(R⁶)₂, or —P(O)(OR⁵)—R⁷—N(R⁵)—C(S)—N(R⁶)₂;
R³ is tetrazole, —C(O)OR⁶ or —C(O)O—R⁷—OC(O)R⁵;
R⁴ is aryl optionally substituted by one or more substituents selected from the group consisting of alkyl, halo, nitro, cyano, —N(R⁶)₂, —R⁷ —N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
each R⁵ is independently hydrogen, alkyl or aralkyl;
each R⁶ is independently hydrogen, alkyl, alkenyl, alkynyl, aryl, aralkyl or aralkenyl;
each R⁷ is a straight or branched alkylene chain (optionally substituted by hydroxy, mercapto, alkylthio, aryl, cycloalkyl, —N(R⁶)₂, —C(O)OR⁶, or—C(O)N(R⁶)₂); and
each R⁸ is independently alkyl, alkenyl, aryl, aralkyl or aralkenyl.

Of this group of compounds of formula (II), preferred compounds are selected from the group consisting of the following:
2-(3-guanidinophenyl)-2-((1-(2-phenylethyl)amino-2-methylpropyl)-(hydroxy)phosphinoyloxy)ethanoic acid;
2-(3-aminophenyl)-2-((phenyl)(hydroxy)phosphinoyloxy)ethanoic acid;
2-(3-guanidinophenyl)-2-((1-amino-2-methylpropyl)(hydroxy)phosphinoyloxy)ethanoic acid; and
2-(3-guanidinophenyl)-2-((1-(benzylaminothiocarbonyl)amino-2-methylpropyl)-(hydroxy)phosphinoyloxy)ethanoic acid.

Of the compounds of formula (II) as set forth above in the Summary of the Invention, another preferred group is that group of compounds of formula (II) wherein:
R¹ is hydrogen;
R² —P(O)(OR⁵)—R⁷—N(R⁵)—S(O)₂—R⁹;
R³ is tetrazole, —C(O)OR⁶ or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
each R⁵ is independently hydrogen, alkyl or aralkyl;
each R⁶ is independently hydrogen, alkyl, alkenyl, alkynyl, aryl, aralkyl or aralkenyl;
each R⁷ is a straight or branched alkylene chain (optionally substituted by hydroxy, mercapto, alkylthio, aryl, cycloalkyl, —N(R⁶)₂, —C(O)OR⁶, or —C(O)N(R⁶)₂);
each R⁸ is independently alkyl, alkenyl, aryl, aralkyl or aralkenyl; and
R⁹ is alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

Of this group of compounds of formula (II), preferred compounds are selected from the group consisting of the following:
2-(3-guanidinophenyl)-2-((1-(benzylsulfonyl)amino-2-methylpropyl)(hydroxy)-phosphinoyloxy)ethanoic acid; and
2-(3-guanidinophenyl)-2-((1-(2-phenylethenylsulfonyl)amino-2-methylpropyl)-(hydroxy)phosphinoyloxy)ethanoic acid.

Of the compounds of formula (II) as set forth above in the Summary of the Invention, another preferred group is that group of compounds of formula (II) wherein:
R¹ is hydrogen;
R² is —P(O)(OR⁵)₂, —P(O)(OR⁵)R⁶, —P(O)(OR⁵)—R⁷—N(R⁶)₂, —P(O)(OR⁵)—R⁷—C(O)—R⁸, —P(O)(OR⁵)—R⁷—N(R⁵)—C(O)OR⁸, —P(O)(OR⁵)—R⁷—N(R⁵)—C(O)—R⁷—N(R⁵)—C(O)OR⁸, —P(O)(OR⁵)—R⁷—N(R⁵)—S(O)₂—R⁹, or —P(O)(OR⁵)—R⁷—N(R⁵)—C(S)—N(R⁶)₂;
R³ is tetrazole;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷ —N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the N-heterocyclyl may be optionally substituted by —C(NR⁵)—N(R⁵)₂, —C(NR⁵)—R⁵, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently cycloalkylene (optionally substituted by alkyl), a straight or branched alkylene chain (optionally substituted by hydroxy, mercapto, alkylthio, aryl, cycloalkyl, —N(R⁶)₂, —C(O)OR⁶, or —C(O)N(R⁶)₂), or a straight or branched alkenylene chain (optionally substituted by hydroxy, mercapto, alkylthio, aryl, cycloalkyl, —N(R⁶)₂, —C(O)OR⁶, or —C(O)N(R⁶)₂);
each R⁸ is independently alkyl, alkenyl, aryl, aralkyl or aralkenyl; and
R⁹ is —R⁷N(R⁶)C(O)OR⁸, haloalkyl, alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), or N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

Of this group of compounds of formula (II), a preferred compound is 2-methyl-1-[1-(3-guanidinophenyl)-1-tetrazolylmethoxy](hydroxy)phosphinoyl-propylcarbamic acid, benzyl ester.

Of the compounds of formula (III) as set forth above in the Summary of the Invention, a preferred group is that group of compounds of formula (III) wherein:
X is —O—;
R² is —P(O)(OR⁵)—R⁷—N(R⁵)—C(O)OR⁸; and
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—N(R⁶)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂).

Of this group of compounds of formula (III), preferred compounds are selected from the group consisting of the following:
2-(3-guanidinophenyl)-2-((1-(benzyloxycarbonyl)aminoethyl)(hydroxy)-phosphinoyloxy)ethanoic acid;
2-(3-guanidinophenyl)-2-(((benzyloxycarbonyl)aminomethyl)(hydroxy)-phosphinoyloxy)ethanoic acid;
2-(3-guanidinophenyl)-2-((1-(benzyloxycarbonyl)amino-2-methylpropyl)(hydroxy)-phosphinoyloxy)ethanoic acid;
2-(3-guanidinophenyl)-2-((1-(benzyloxycarbonyl)aminohexyl)(hydroxy)-phosphinoyloxy)ethanoic acid;
2-(3-aminophenyl)-2-((1-(benzyloxycarbonyl)amino-2-methylpropyl)-(hydroxy)phosphinoyloxy)ethanoic acid,
2-(3-guanidinophenyl)-2-((1-(benzyloxycarbonyl)amino-3-methylbutyl)-(hydroxy)phosphinoyloxy)ethanoic acid;
2-(2-chloro-3-guanidinophenyl)-2-((1-(benzyloxycarbonyl)amino-2-methylpropyl)-(hydroxy)phosphinoyloxy)ethanoic acid;
2-(3-guanidinophenyl)-2-((1-(benzyloxycarbonyl)amino-1-phenylmethyl)-(hydroxy)phosphinoyloxy)ethanoic acid;
2-(2-fluoro-3-guanidinophenyl)-2-((1-(benzyloxycarbonyl)amino-2-methylpropyl)-(hydroxy)phosphinoyloxy)ethanoic acid;
2-(3-guanidinophenyl)-2-((1-(benzyloxycarbonyl)amino-1-cyclohexylmethyl)-(hydroxy)phosphinoyloxy)ethanoic acid;
2-(2-methyl-3-guanidinophenyl)-2-((1-(benzyloxycarbonyl)amino-2-methylpropyl)-(hydroxy)phosphinoyloxy)ethanoic acid;
2-(3-(amino)methylphenyl)-2-((1-(benzyloxycarbonyl)amino-2-methylpropyl)-(hydroxy)phosphinoyloxy)ethanoic acid;
2-(3-(guanidinomethyl)phenyl)-2-((1-(benzyloxycarbonyl)amino-2-methylpropyl)-(hydroxy)phosphinoyloxy)ethanoic acid;
2-(3-(1-iminoethylaminophenyl))-2-((1-(benzyloxycarbonyl)amino-2-methylpropyl)-(hydroxy)phosphinoyloxy)ethanoic acid;
2-(3-(t-butoxycarbonylamino)methylphenyl)-2-((1-(benzyloxycarbonyl)amino-2-methylpropyl)(hydroxy)phosphinoyloxy)ethanoic acid;
2-(3-(ethoxycarbonylamino)methylphenyl)-2-((1-(benzyloxycarbonyl)amino-2-methylpropyl)(hydroxy)phosphinoyloxy)ethanoic acid;
2-(3-(isopropoxycarbonylamino)methylphenyl)-2-((1-(benzyloxycarbonyl)amino-2-methylpropyl)(hydroxy)phosphinoyloxy)ethanoic acid;
2-(3-(2,2-dimethylpropylcarbonylamino)methylphenyl)-2-((1-(benzyloxycarbonyl)-amino-2-methylpropyl)(hydroxy)phosphinoyloxy)ethanoic acid;
2-(3-guanidinophenyl)-2-((1-(2-phenylethylcarbonyl)amino-2-methylpropyl)-(hydroxy)phosphinoyloxy)ethanoic acid; and
2-(3-guanidinophenyl)-2-((1-(2-phenylethenylcarbonyl)amino-2-methylpropyl)-(hydroxy)phosphinoyloxy)ethanoic acid.

Of the compounds of formula (III) as set forth above in the Summary of the Invention, another preferred group is that group of compounds of formula (III) wherein:
X is —O—;
R² is —P(O)(OR⁵)—R⁷—N(R⁵)—C(O)—R⁷—N(R⁵)—C(O)OR⁸; and
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—N(R⁶)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂).

Of this group of compounds of formula (III), preferred compounds are selected from the group consisting of the following:
2-(3-guanidinophenyl)-2-[(1-(1-benzyloxycarbonylamino-2-(4-hydroxyphenyl)-ethylcarbonyl)amino-2-methylpropyl)(hydroxy)phosphinoyloxy]ethanoic acid;
2-(3-guanidinophenyl)-2-[(1-(1-benzyloxycarbonylamino-2-phenylethylcarbonyl)amino-2-methylpropyl)(hydroxy)phosphinoyloxy]ethanoic acid;
2-(2-fluoro-3-guanidinophenyl)-2-[(1-(1-benzyloxycarbonylamino-2-phenyl-ethylcarbonyl)amino-2-methylpropyl)(hydroxy)phosphinoyloxy]ethanoic acid;
2-(3-guanidinophenyl)-2-[(1-(1-phenylcarbonylamino-2-phenylethylcarbonyl)amino-2-methylpropyl)(hydroxy)phosphinoyloxy]ethanoic acid;
2-(3-guanidinophenyl)-2-[(1-(1-ethoxycarbonylamino-2-phenylethylcarbonyl)amino-2-methylpropyl)(hydroxy)phosphinoyloxy]ethanoic acid;
2-(3-guanidinophenyl)-2-[(1-(1-benzyloxycarbonylamino-3-phenylpropyl-carbonyl)amino-2-methylpropyl)(hydroxy)phosphinoytoxy]ethanoic acid; and
2-(3-(amino)methylphenyl)-2-[(1-(1-benzyloxycarbonylamino-3-phenylpropyl-carbonyl)amino-2-methylpropyl)(hydroxy)phosphinoyloxy]ethanoic acid.

Of the compounds of formula (III) as set forth above in the Summary of the Invention, another preferred group is that group of compounds of formula (III) wherein:
X is —CH₂—;
R² is —P(O)(OR⁵)—R⁷—N(R⁵)—C(O)R⁶ or —P(O)(OR⁵)—R⁷—N(R⁵)—C(O)OR⁸; and
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—N(R⁶)—N(R⁶)₂, —C(O)—R⁷—N(R⁶)₂,
—N(R⁵)—C(N R⁵)—N(R⁵)₂, —N(R⁵)—C(O)—N(R⁶)₂ and —N(R⁵)—C(O)—R⁷—N(R⁶₂).

Of this group of compounds of formula (III), preferred compounds are selected from the group consisting of the following:
2-(3-(amino)methylphenyl)-3-((1-(methylcarbonyl)amino-2-methylpropyl)(hydroxy)-phosphinoyl)propanoic acid;
2-(3-(hydrazinocarbonyl)phenyl)-3-((1-(benzyloxycarbonyl)amino-2-methylpropyl)-(hydroxy)phosphinoyl)propanoic acid;
2-(3-guanidinophenyl)-3-((1-(benzyloxycarbonyl)amino-3-methylbutyl)-(hydroxy)phosphinoyl)propanoic acid;
2-(3-guanidinophenyl)-3-(((benzyloxycarbonyl)aminomethyl)(hydroxy)-phosphinoyl)propanoic acid; 2-(3-guanidinophenyl)-3-((1-(benzyloxycarbonyl)aminoethyl)(hydroxy)-phosphinoyl)propanoic acid;
2-(3-guanidinophenyl)-3-((1-(benzyloxycarbonyl)amino-2-methylpropyl)(hydroxy)-phosphinoyl)propanoic acid;
2-(2-chloro-5-guanidinophenyl)-3-((1-(benzyloxycarbonyl)amino-2-methylpropyl)(hydroxy)phosphinoyl)propanoic acid;
2-(3-(amino)methylphenyl)-3-((1-(benzyloxycarbonyl)amino-2-methylpropyl)(hydroxy)-phosphinoyl)propanoic acid; and
2-(3-(amino)methylphenyl)-3-((1-(2-phenylethylcarbonyl)amino-2-methylpropyl)(hydroxy)phosphinoyl)propanoic acid.

Of the compounds of formula (III) as set forth above in the Summary of the Invention, another preferred group is that group of compounds of formula (III) wherein:
X is —CH₂—;
R² is —P(O)(OR⁵)—R⁷—N(R⁵)—C(O)—R⁷—N(R⁵)—C(O)OR⁸; and
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—N(R⁶)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂).

Of this group of compounds of formula (III), preferred compounds are selected from the group consisting of the following:
2-(3-guanidinophenyl)-3-(((1-benzyloxycarbonylamino-2-phenylethyl)-carbonylaminomethyl)(hydroxy)phosphinoyl)propanoic acid; and
2-(3-guanidinophenyl)-3-(((1-benzyloxycarbonylamino-2-phenylethyl)-carbonylaminomethyl)(hydroxy)phosphinoyl)propanoic acid.

### EXAMPLES

The TAFI inhibitors of the invention were tested in four in vivo efficacy models to demonstrate the usefulness of the pharmaceutical compositions of the invention in potentiating fibrinolysis, which in turn enhances myocardial reperfusion and facilitates PCI in patients with STEMI: 1) a rat femoral artery thrombosis model; 2) a dog femoral artery thrombosis model; 3) a rabbit jugular vein thrombosis model; and 4) a rat lung fibrin deposition model.

### Example 1

### Rat Femoral Artery Thrombosis Model

A rat femoral artery thrombosis model was used to demonstrate that TAFI inhibition by i.v. administration of a potent and selective TAFI inhibitor would potentiate fibrinolysis. A stable, occlusive thrombus was formed by giving rose Bengal intravenously followed by light irradiation. Free radicals generated at the site of irradiation result in endothelial damage that causes an occlusive thrombus. Thrombotic occlusion and effective fibrinolysis (reperfusion) were monitored by measuring blood flow with a Doppler flow probe. Since no spontaneous reperfusion is observed in this model, a threshold dose of tPA was used to mimic the endogenous fibrinolytic activity observed in patients with STEMI undergoing PCI (16-25% spontaneous reperfusion rate). The threshold dose of tPA chosen resulted in a low reperfusion rate of 13%. Based on the results of phanmacokinetic studies and in vitro clot lysis assays, two doses (3 and 10 mg/kg) of a TAFI inhibitor of the invention, (2S)-2-(3-(amino)methylphenyl)-3-(((1R)-1-(3-phenylpropylsulfonyl)amino-2-methylpropyl)(hydroxy)phosphinoyl)propanoic acid trifluoroacetate, were used.

Male Wistar rats (241-270 g) were anesthetized with sodium pentobarbital. A section of the right femoral artery was isolated carefully, and a pulse Doppler flow probe was placed for monitoring blood flow. The left carotid artery, both jugular veins, and left femoral vein were cannulated with polyethylene tubes for monitoring blood pressure and heart rate, for injection of tPA, rose Bengal and TAFI inhibitor, respectively. Blood flow, blood pressure and heart rate were continuously recorded on a thermal recorder during the experiment. An occlusive thrombus was induced in the right femoral artery by bolus injection of rose Bengal followed by light exposure. Occlusive thrombus was observed approximately 6 min after the injection of rose Bengal and start of irradiation.

tPA Dose Response Study:

Rats were assigned to four groups for the tPA dose response as follows:
(1) Vehicle (saline; n=8)
(2) tPA (72 μg/kg/min=2.5 million I.U./kg/hr; n=8)
(3) tPA (115 μg/kg/min=4 million I.U./kg/hr; n=10)
(4) tPA (230 μg/kg/min=8 million I.U./kg/hr; n=8)

tPA or vehicle was infused via the right jugular vein beginning 10 min after the formation of occlusive thrombus and the infusion was continued for 60 min. The incidence of reperfusion for the vehicle group and the 72, 115 and 230 μg/kg/min tPA treated groups were 0/8, 1/8, 5/10 and 6/8, respectively. tPA dose-dependently increased reperfusion incidence (p<0.01, Cumulative chi-square test). This study demonstrated that the incidence of reperfusion was a useful measure for evaluating the effects of thrombolytic agents in this model. The results demonstrated that tPA at 72 μg/kg/min was a threshold fibrinolytic dose and tPA at 230 μg/kg/min was a sub-maximal dose.

TAFI Inhibitor Efficacy Study:

Rats were assigned to three groups for the TAFI inhibitor efficacy study as follows:
(1) tPA threshold (72 μg/kg/min, n=16)
(2) low TAFI inhibitor (3 mg/kg+0.03 mg/kg/min) and tPA threshold (72 μg/kg/min) (n=8)
(3) high TAFI inhibitor (10 mg/kg+0.10 mg/kg/min) and tPA threshold (72 μg/kg/min) (n=8)

Administration of TAFI inhibitor or vehicle via the left femoral vein was started simultaneously with tPA via the right jugular vein beginning 10 min after the formation of occlusive thrombus, both given as a bolus over 1 min and infusion for 59 min. At the end of the infusion, blood was collected from the inferior vena cava for assays of plasma drug concentration. The incidence of reperfusion was used as the index of drug efficacy.

The incidence of reperfusion for the tPA threshold dose (72 μg/kg/min) group, low TAFI inhibitor dose (3 mg/kg+0.03 mg/kg/min)+tPA threshold dose group, and high TAFI inhibitor dose (10 mg/kg+0.10 mg/kg/min)+tPA threshold dose group were 2/16, 2/8 and 5/8, respectively (Table 1). The combination of high dose TAFI inhibitor+tPA threshold dose significantly increased reperfusion incidence compared with the tPA threshold dose alone (p=0.02, Fisher exact test). This enhanced fibrinolytic activity was similar to that of the high dose tPA alone (between 115 and 230 μg/kg/min).

TABLE 1
Summary of Rat Thrombosis Model Results

| Rat Groups | Incidence of reperfusion |
|---|---|
| Threshold dose tPA (72 μg/kg/min) | 13% (2/16) |
| 3 mg/kg TAFI inhibitor + tPA post-thrombus | 25% (2/8) p = ns |
| formation | |
| 10 mg/kg TAFI inhibitor + tPA post-thrombus | 63% (5/8) p < 0.03 |
| formation | |

A TAFI inhibitor of the invention, when tested in this model, demonstrated the ability to enhance fibrinolysis in vivo.

### Example 2

### Dog Femoral Artery Thrombosis Model

A dog femoral artery thrombosis model was used to test that TAFI inhibition by i.v. administration of a potent and selective TAFI inhibitor would potentiate fibrinolysis. A stable, occlusive thrombus was formed using FeCl₂ to chemically injure the arterial wall. Thrombotic occlusion and reperfusion (effective fibrinolysis) were monitored by measuring blood flow with a Doppler flow probe. Since no spontaneous reperfusion is observed in this model, a sub-maximal dose of tPA was used to enhance baseline activity. Based on the results of pharmacokinetic studies and in vitro clot lysis assays, two doses (1 and 10 mg/kg) of a TAFI inhibitor of the invention, (2S)-2-(3-(amino)methylphenyl)-3-(((1R)-1-(3-phenylpropylsulfonyl)amino-2-methylpropyl)(hydroxy)phosphinoyl)propanoic acid trifluoroacetate, were used. Bleeding propensity was measured by determining the time required for bleeding to stop following a series of standardized wounds (sectioning of the cuticle).

Male Beagle dogs (8-12 kg) were anesthetized with isoflurane (2-3%). The femoral artery was isolated and a Transonic flow probe (2.5 SB) placed around the vessel. The left and right jugular veins were catheterized for tPA and TAFI inhibitor/vehicle administration. Femoral artery blood flow, systemic blood pressure, heart rate and breathing patterns were monitored and recorded on a data acquisition system during the experiment. Bleeding was induced by sectioning of the cuticle (approximately 3 mm from the extremity) of the left forepaw nail using a nail trimmer. Blood was blotted on a strip of filter paper. The time for the cut to cease bleeding was recorded as the primary bleeding time.

After stabilization of hemodynamic parameters, a blood sample was taken for drug levels. A filter paper (5×6 mm), soaked in 10% FeCl₂, was applied on the isolated femoral artery for 10 minutes to chemically injure the endothelium locally. This caused gradual formation of a thrombotic occlusion inside the vessel, which completely occluded the femoral artery (blood flow=0). A second blood sample was collected 25 min after occlusion. tPA was injected as a bolus ( 1/10 of the total dose) followed by a 30 min-infusion (9/10 of the total dose) starting 40 min after occlusion. Bleeding time was determined and a blood sample was collected 20 min after the start of the tPA infusion. All experimental observations continued for 60 min, at which time the tPA infusion was stopped and a final blood sample was collected.

A dose of tPA (10 μg/kg/min) was used in all animals to enhance baseline fibrinolytic activity. In one group of dogs (Pre), TAFI inhibitor or vehicle (saline) was administered 10 minutes before FeCl₂ application. In another group (Post), TAFI inhibitor was administered 30 min after femoral artery occlusion. TAFI inhibitor (1 or 10 mg/kg) or saline (1 ml/kg) was injected as a bolus followed by a continuous infusion (0.2 or 2 mg/kg/h at a rate of 25 μl/kg/min).

Plasma drug concentration was measured using LC/MS/MS techniques. Ex vivo α2-antiplasmin, an indicator of systemic plasmin activation, was measured by colorimetric assay. Exogenously activated TAFI (10 nM) was added into the blank plasma and TAFI activity measured by colorimetric assays was defined as 100%. Plasma from TAFI inhibitor-administered dogs were treated in the same way, and results were expressed as the percent inhibition of the spiked TAFI activity.

Bleeding time was prolonged dose-dependently by tPA (Table 2). The dose of 10 μg/kg/min of tPA induced reperfusion in 50% of the dogs, which narrowed the dynamic range to detect a significant treatment effect. Combined with 10 μg/kg of tPA, TAFI inhibitor at 10 mg/kg given before induction of thrombus increased reperfusion rate to 100% of dogs. This significant increase in incidence of reperfusion was without further prolongation of bleeding time. However, TAFI inhibitor pre-treatment did not significantly affect the time to occlusion induced by FeCl₂ (31.1±3.9 min, n=6, p=ns) compared to the control group (38.6±2.7 min, n=10).

TAFI inhibitor given after the induction of thrombus at 1 mg/kg had no effect on the incidence of reperfusion (50%, n=6). The 10 mg/kg dose administered after thrombus formation increased reperfusion from 50% to 67%; however, the differences were not statistically significant (n=6) (Table 2).

The activity of α2-antiplasmin, an indicator of systemic plasmin activation, was dose-dependently reduced by tPA. TAFI inhibitor administered pre- or post-thrombus formation did not change the inhibition of α2-antiplasmin activity induced by tPA infusion.

TABLE 2
Summary of Dog Thrombosis Model Results

| Dog Groups | Incidence of reperfusion |
|---|---|
| Threshold dose tPA (10 μg/kg/min) | 50% (5/10) |
| 1 mg/kg TAFI inhibitor + tPA post-thrombus | 50% (3/6) p = ns |
| formation | |
| 10 mg/kg TAFI inhibitor + tPA post-thrombus | 67% (4/6) p = ns |
| formation | |
| 10 mg/kg TAFI inhibitor + tPA pre-thrombus | 100% (6/6) p < 0.05 |
| formation | |

A TAFI inhibitor of the invention, when tested in this model, demonstrated the ability to enhance fibrinolysis in vivo.

### Example 3

### Rabbit Jugular Vein Thrombosis Model

A rabbit jugular vein thrombosis model was used to demonstrate that TAFI inhibition with a potent and selective TAFI inhibitor in combination with tPA would enhance fibrinolysis. Thrombosis was initiated ex vivo and introduced into the jugular vein. Treatment compound(s) were administered to the animals after thrombus formation and introduction. Fibrinolysis was assessed by measuring clot weight at the end of the study. Pilot studies indicated that enhancement of fibrinolysis by a TAFI inhibitor combined with a threshold dose of tPA was similar to that observed with a 3-fold higher dose of tPA alone. The threshold dose of tPA was combined with either a TAFI inhibitor of the invention, (2S)-2-(3-(amino)methylphenyl)-3-(((1R)-1-(3-phenylpropylsulfonyl)amino-2-methylpropyl)(hydroxy)phosphinoyl)propanoic acid trifluoroacetate, or the peptidic TAFI inhibitor CPI, which was used as a positive control to confirm the effect of TAFI inhibition in this model.

Male New Zealand white rabbits (2.35-3.14 kg) were anesthetized using isoflurane. A segment of the jugular vein was isolated with ligatures and injected with a mixture of autologous citrated blood and thromboplastin to induce thrombus formation. The thrombus formed in the isolated venous segment was allowed to mature for 30 minutes prior to releasing the ligatures and initiating thrombolytic treatment.

The doses of tPA and CPI used for this study were based on four previous studies, which demonstrated that tPA (10 μg/kg+67 μg/kg/hr) was a threshold fibrinolytic dose and a three-fold higher dose of tPA (30 μg/kg+200 μg/kg/hr) was a maximally effective dose.

Rabbits were divided into the following treatment groups (10-12 animals per group):
(1) vehicle; phosphate buffered saline (PBS)
(2) threshold fibrinoloytic dose tPA (10 μg/kg+67 μg/kg/hr)
(3) CPI (500 μg/kg+300 μg/kg/hr)+tPA low (10 μg/kg+67μg/kg/hr)
(4) low TAFI inhibitor (0.3 mg/kg+0.14 mg/kg/hr)+tPA low (10 μg/kg+67 μg/kg/hr)
(5) high TAFI inhibitor (3 mg/kg+1.4 mg/kg/hr)+tPA low (10 pg/kg+67 μg/kg/hr)
(6) maximally effective dose tPA (30 μg/kg+200 μg/kg/hr)

Blood samples were collected from the left jugular vein for measurement of TAFI inhibitor plasma drug levels prior to releasing the ligatures (time=0), and at 1, 10, 30, 60, and 90 minutes after starting thrombolytic treatment. CPI, TAFI inhibitor, tPA or vehicle were administered as bolus injections, followed by a constant infusion for 90 minutes via the right marginal ear vein. At the end of 90 minutes, the rabbits were euthanized and the thrombus was removed for determination of wet and dry weights. The relative effects of all treatment groups were the same, regardless of whether wet or dry weights were utilized for analysis, so only wet weights are used in the subsequent discussion. The assessment of bleeding risk was evaluated by measuring the weight of blood loss from toe nail clips performed prior to thrombolytic treatment, and at the end of the study. Re-bleeding from the previous site following initial hemostasis was also recorded.

Data were analyzed using ANOVA followed by the LSD test for a comparison of differences in thrombus weight in the various treatment groups. In the vehicle-treated animals (group 1) thrombus weights averaged 98±9 mg. In the low dose tPA-treated animals (group 2), thrombus weights tended to be lower (80±11 mg); however, the change was not significantly different from the vehicle-treated animals. When either CPI (group 3) or TAFI inhibitor (group 4 or 5) was given together with the low dose of tPA, the threshold effect of tPA was enhanced, and a significant reduction in thrombus weights was observed when compared to the vehicle-treated group. This enhancement of fibrinolytic activity of tPA for the CPI group (68±10 mg) and the low dose TAFI inhibitor group (63±10 mg) was similar to results obtained with the high dose TAFI inhibitor group (56±13 mg). In animals treated with the higher dose of tPA (group 6), a significant reduction in thrombus weight (41±7 mg) was observed compared to both the vehicle and low dose tPA groups.

TABLE 3
Summary of Rabbit Thrombosis Model Results

| | % Reduction |
|---|---|
| | Thrombus |
| Rabbit Groups | Weight |
| Threshold dose tPA (10 μg/kg + 1 μg/kg/min) | 18% |
| 0.3 mg/kg TAFI inhibitor + tPA post-thrombus formation | 36% p = ns |
| 3 mg/kg TAFI inhibitor + tPA post-thrombus formation | 43% p = ns |

In conclusion, combination of TAFI inhibitor or CPI with a threshold dose of tPA resulted in enhanced fibrinolysis. The magnitude of the effect was similar to that observed with a 3-fold higher dose of tPA. In this model, although there was considerable variability in thrombus weight, the data demonstrate that TAFI inhibition with a potent and selective TAFI inhibitor (TAFI inhibitor) enhanced fibrinolytic activity of a threshold dose of tPA.

### Example 4

### Rat Lung Fibrin Deposition Model

This model was used to test the hypothesis that inhibition of endogenous TAFI can enhance fibrinolysis. Batroxobin, a thrombin-like enzyme, was used to hydrolyze fibrinogen to fibrin, which deposits in tissues, especially in the lungs. Following tissue deposition, fibrin is lysed into fibrin degradation products, which return to the blood. In this study, ¹²⁵I-labeled fibrinogen was given and tissue accumulation of fibrin and the subsequent fibrinolysis were assessed by the mobilization of ¹²⁵I from blood to tissue (generation of ¹²⁵I-fibrin from ¹²⁵I-fibrinogen) and back to blood (generation of ¹²⁵I-fibrin degradation products from ¹²⁵I-fibrin) after batroxobin injection. LPS has been known to stinulate the release of numerous endogenous factors that can inhibit fibrinolytic activity, including TAFI, thus preventing spontaneous fibrinolysis in the lungs.

Male Sprague-Dawley rates (300-350g) were randomly divided into three groups:
(1) untreated (controls)
(2) treated with LPS
(3) treated with LPS+TAFI inhibitor.
   Animals assigned to receive LPS (0.5 mg/kg, iv) were injected 3 hours before batroxobin injection. Twenty minutes before batroxobin injection, all animals were anesthetized with pentobarbital (65 mg/kg, ip), and saline-filled catheters were placed into the right carotid artery and left jugular vein for blood collection and drug injection, respectively. ¹²⁵I-fibrinogen (1 mCi) was administered to all animals 10 min. prior to batroxobin injection and animals assigned to receive the TAFI inhibitor (10 mg/kg) were injected 5 min. before the batroxobin. The rats were sacrificed at either 5 or 30 min after batroxobin injection.

With a TAFI inhibitor of the invention, (2S)-2-(3-(amino)methylphenyl)-3-(((1R)-1-(3-phenylpropylsulfonyl)amino-2-methylpropyl)(hydroxy)phosphinoyl)propanoic acid trifluoroacetate, tested in this model, the following results were obtained: In the controls, intravenous injection of batroxobin induced a rapid mobilization of ˜80% of the circulating ¹²⁵I to tissues at 5 min. A significant fraction of the displaced ¹²⁵I from the blood was lodged in the lungs, as shown by an increase in CPM ratio at 5 min. At 30 min after batroxobin injection, ˜50% of displaced iodine returned to circulation and CPM in the lungs had been reduced to ˜10% of its value at 5 min, indicating a rapid lysis of the fibrin accumulated in the lungs.

In LPS-treated rats, blood reactivity at 5 min was slightly higher than in blood from control animals, without major impact on the fraction of mobilized ¹²⁵I to tissues. At 5 min after batroxobin injection, no difference in radioactivity was found in the lungs from LPS-treated and control rats. However, a significant reduction in blood radioactivity associated with higher counts in the lungs were detected in LPS-treated rats at 30 min, indicating impaired fibrinolysis.

The TAFI inhibitor co-administered to LPS-treated rats did not change the radioactive readings in either blood or lungs at 5 min, in relation to LPS-treated rats. However, the TAFI inhibitor prevented LPS-induced recovery of blood CPM and resulted in ¹²⁵I retention in the lungs at 30 min. Indeed, CPM in the lungs of control and LPS+TAFI inhibitor-treated rats was not different from each other at 30 min., indicating that the TAFI inhibitor of the invention is effective in preventing LPS-induced impairment of fibrinolysis.

What is claimed is:

1. A method of enhancing myocardial reperfusion and facilitating PCI comprising administering to a patient in need thereof a therapeutically effective amount of a compound of formula (I) or a pharmaceutically acceptable salt thereof: 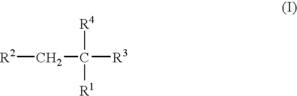
wherein:
R¹ is hydrogen, alkyl, alkenyl, aralkyl, or aralkenyl;
R² is —SH, —S—C(O)—R⁸, —P(O)(OR⁵)₂, —P(O)(OR⁵)R⁶, —P(O)(OR⁵)—R⁷—N(R⁶)₂, —P(O)(OR⁵)—R⁷—C(O)—R⁸, —P(O)(OR⁵)—R⁷—N(R⁵)—C(O)OR⁸, —P(O)(OR⁵)—R⁷—N(R⁵)—C(O)—R⁷—N(R⁵)—C(O)OR⁸, —P(O)(OR⁵)—R⁷—N(R⁵)—S(O)₂—R⁹, or —P(O)(OR⁵)—R⁷—N(R⁵)—C(S)—N(R⁶)₂;
R³ is tetrazole, —C(O)OR⁶, —C(O)O—R⁷—OC(O)R⁵, —S(O)OR⁵, —S(O)₂OR⁵, —P(O)(OR⁵)₂, —P(O)(OR⁵)R⁶, or —B(OR⁵)₂;
R⁴ is aryl optionally substituted by one or more substituents selected from the group consisting of alkyl, halo, haloalkyl, haloalkoxy, mercapto, alkylthio, phenyl, cycloalkyl, nitro, cyano, —OR⁶, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(N R⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the N-heterocyclyl may be optionally substituted by —C(NR⁵)—N(R⁵)₂, —C(NR⁵)—R⁵, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently cycloalkylene (optionally substituted by alkyl), a straight or branched alkylene chain (optionally substituted by hydroxy, mercapto, alkylthio, aryl, cycloalkyl, —N(R⁶)₂, —C(O)OR⁶, or —C(O)N(R⁶)₂), or a straight or branched alkenylene chain (optionally substituted by hydroxy, mercapto, alkylthio, aryl, cycloalkyl, —N(R⁶)₂, —C(O)OR⁶, or —C(O)N(R⁶)₂);
each R⁸ is independently alkyl, alkenyl, aryl, aralkyl or aralkenyl; and
R⁹ is —R⁷N(R⁶)C(O)OR⁸, haloalkyl, alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or—N(R⁶)C(O)R⁶), or N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶);
provided that when R³ is —C(O)OH or when R⁴ is a substituted aryl or substituted N-heterocyclyl,
R² can not be —P(O)(OR⁵)—R⁷—N(H)—C(O)OR⁸ or
—P(O)(OR⁵)—R⁷—N(H)—C(O)—R⁷—N(R⁵)—C(O)OR⁸;
as a single stereoisomer, a mixture of stereoisomers, or as a racemic mixture of stereoisomers.

2. The method of claim 1 wherein:
R¹ is hydrogen;
R is —SH or —S—C(O)—R⁸;
R³ is tetrazole, —C(O)OR⁶ or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR¹¹, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R is independently hydrogen, alkyl, alkenyl, alkynyl, aryl, aralkyl or aralkenyl;
each R⁷ is independently a straight or branched alkylene chain optionally substituted by hydroxy, mercapto, alkylthio, aryl, cycloalkyl, —N(R⁶)₂, —C(O)OR⁶, or —C(O)N(R⁶)₂; and
each R⁸ is independently alkyl, alkenyl, aryl, aralkyl or aralkenyl.

3. The method of claim 2 wherein:
R¹ is hydrogen;
R² is —SH or —S—C(O)—R⁸;
R is —C(O)OR⁶;
R⁴ is aryl optionally substituted by one or more substituents selected from the group consisting of halo, nitro, —N(R⁶)₂, —R⁷—N(R⁶)₂ and —N(R⁵)—C(NR⁵)—N(R⁵)₂;
each R⁵ is independently hydrogen, alkyl or aralkyl;
each R⁶ is independently hydrogen, alkyl, aryl or aralkyl;
R⁷ is a straight or branched alkylene chain; and
R⁸ is alkyl, alkenyl, aryl, aralkyl or aralkenyl.

4. The method of claim 3 wherein the compound of formula (i) is selected from the group consisting of:
2-(4-guanidinophenyl)-3-mercaptopropanoic acid;
2-(3-guanidinophenyl)-3-mercaptopropanoic acid;
2-(3-aminophenyl)-3-mercaptopropanoic acid; and
2-(2-chloro-5-guanidinophenyl)-3-mercaptopropanoic acid.

5. The method of claim 2 wherein:
R¹ is hydrogen;
R is —SH, or —S—C(O)—R⁸;
R is —C(O)OR⁶;
R⁴ is 3(4)-piperidinyl wherein the nitrogen atom in the piperidinyl radical is optionally substituted by —C(NR⁵)—N(R⁵)₂, —C(N R⁶)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, aryl or aralkyl;
R⁷ is a straight or branched alkylene chain optionally substituted by hydroxy, mercapto, alkylthio, aryl, cycloalkyl, —N(R⁶)₂, —C(O)OR⁶, or —C(O)N(R⁶)₂; and
R⁸ is independently alkyl, alkenyl, aryl, aralkyl or aralkenyl.

6. The method of claim 5 wherein the compound of formula (I) is selected from the group consisting of:
2-(piperidin4-yl)-3-mercaptopropanoic acid;
2-(1-amidinopiperidin-4-yl)-3-mercaptopropanoic acid;
2-(1-(1-iminoethyl)piperidin-4-yl)-3-mercaptopropanoic acid;
2-(1-(aminomethylcarbonyl)piperidin-4-yl)-3-mercaptopropanoic acid;
2-(piperidin-3-yl)-3-mercaptopropanoic acid; and
2-(1-amidinopiperidin-3-yl)-3-mercaptopropanoic acid.

7. The method of claim 1 wherein:
R¹ is hydrogen;
R² is —P(O)(OR⁵)₂, —P(O)(OR⁵)R⁶ or —P(O)(OR⁵)—R⁷—C(O)—R⁸;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂ —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain optionally substituted by hydroxy, mercapto, alkylthio, aryl, cycloalkyl, —N(R⁶)₂, —C(O)OR⁶, or —C(O)N(R⁶)₂; and
each R⁸ is independently alkyl, alkenyl, aryl, aralkyl or aralkenyl.

8. The method of claim 7 wherein:
R¹ is hydrogen;
R² is —P(O)(OR⁵)₂, —P(O)(OR⁵)R⁶ or —P(O)(OR⁵)—R⁷—C(O)—R⁸;
R³ is —C(O)OR⁶;
R⁴ is aryl optionally substituted by one or more substituents selected from the group consisting of halo, nitro, —N(R⁶)₂, —R⁷—N(R⁶)₂ and —N(R⁵)—C(NR⁵)—N(R⁵)₂;
each R⁵ is independently hydrogen, alkyl or aralkyl;
each R⁶ is independently hydrogen, alkyl, aryl or aralkyl;
each R⁷is independently a straight or branched alkylene chain optionally substituted by aryl, —N(R⁶)₂ or —C(O)OR⁶; and
R⁸ is alkyl, alkenyl, aryl, aralkyl or aralkenyl.

9. The method of claim 8 wherein the compound of formula (I) is selected from the group consisting of:
2-(3-guanidinophenyl)-3-phosphonopropanoic acid;
2-(3-aminophenyl)-3-((phenyl)(hydroxy)phosphinoyl)propanoic acid;
2-(3-aminophenyl)-3-((4-phenylbutyl)(hydroxy)phosphinoyl)propanoic acid;
2-(3-aminophenyl)-3-((pentyl)(hydroxy)phosphinoyl)propanoic acid;
2-(3-guanidinophenyl)-3-((phenyl)(hydroxy)phosphinoyl)propanoic acid;
2-(3-guanidinophenyl)-3-((4-phenylbutyl)(hydroxy)phosphinoyl)propanoic acid;
2-(3-guanidinophenyl)-3-((pentyl)(hydroxy)phosphinoyl)propanoic acid;
2-(3-guanidinophenyl)-3-((4-methylpentyl)(hydroxy)phosphinoyl)propanoic acid;
2-(3-guanidinophenyl)-3-((3-phenylpropyl)(hydroxy)phosphinoyl)propanoic acid;
2-(3-guanidinophenyl)-3-((3-phenylprop-2-enyl)(hydroxy)phosphinoyl)propanoic acid;
2-(3-guanidinophenyl)-3-((phenylmethyl)(hydroxy)phosphinoyl)propanoic acid;
2-(3-guanidinophenyl)-3-((pentyl)(hydroxy)phosphinoyl)propanoic acid methyl ester;
2-(3-guanidinophenyl)-3-((ethyl)(hydroxy)phosphinoyl)propanoic acid;
2-(3-guanidinophenyl)-3-((2-phenylethyl)(hydroxy)phosphinoyl)propanoic acid; and
2-(3-guanidinophenyl)-3-((2-(methylcarbonyl)ethyl)(hydroxy)phosohinoyl)propanoic acid.

10. The method of claim 1 wherein:
R¹ is hydrogen;
R² is —P(O)(OR⁵)—R⁷—N(R⁵)—C(O)OR⁸;
R³ is —C(O)OR⁶ (where R⁶ is alkyl, aryl or aralkyl);
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂) where each R⁶ is independently hydrogen, alkyl, aryl or aralkyl;
each R⁵ is independently hydrogen, alkyl or aralkyl;
each R is a straight or branched alkylene chain optionally substituted by aryl, —N(R⁶)₂ or —C(O)OR⁶; and
each R⁸ is independently alkyl, alkenyl, aryl, aralkyl or aralkenyl.

11. The method of claim 10 wherein the compound of formula (I) is selected from the group consisting of:
2-(3-(t-butoxycarbonylamino)methylphenyl)-3-((1-(benzyloxycarbonyl)amino-2-methylpropyl)(hydroxy)phosphinoyl)propanoic acid t-butyl ester; and
2-(3-(t-butoxycarbonylamino)methylphenyl)-3-((1-(benzyloxycarbonyl)amino-2-methylpropyl)(ethoxy)phosphinoyl)propanoic acid t-butyl ester.

12. The method of claim 1 wherein:
R¹ is hydrogen;
R²is —P(O)(OR⁵)—R⁷—N(R⁶)₂ or —P(O)(OR⁵)—R⁷—N(R⁵)—C(S)—N(R⁶)₂;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
each R⁵ is independently hydrogen, alkyl or aralkyl;
each R⁶ is independently hydrogen, alkyl, alkenyl, alkynyl, aryl, aralkyl or aralkenyl;
each R⁷is a straight or branched alkylene chain optionally substituted by hydroxy, mercapto, alkylthio, aryl, cycloalkyl, —N(R⁶)₂, —C(O)OR⁶, or —C(O)N(R⁶)₂; and
each R⁸ is independently alkyl, alkenyl, aryl, aralkyl or aralkenyl.

13. The method of claim 12 wherein the compound of formula (I) is selected from the group consisting of:
2-(3-(t-butoxycarbonylamino)methylphenyl)-3-((1-amino-2-methylpropyl)(hydroxy)-phosphinoyl)propanoic acid t-butyl ester; and
2-(3-(t-butoxycarbonylamino)methylphenyl)-3-((1-amino-2-methylpropyl)(ethoxy)-phosphinoyl)propanoic acid t-butyl ester.

14. The method of claim 1 wherein:
R¹ is hydrogen;
R² is —P(O)(OR⁵)—R⁷—N(R⁵)—S(O)₂—R⁹;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸ —N(R⁶)—C(O)—R⁶, —R⁷—N(R⁶)—C(O)—R⁶, —C(O)—N(R⁶)₂, —C(O)—R⁷ —N(R)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷is independently cycloalkylene (optionally substituted by alkyl), a straight or branched alkylene chain (optionally substituted by hydroxy, mercapto, alkylthio, aryl, cycloalkyl, —N(R⁶)₂, —C(O)OR⁶, or —C(O)N(R⁶)₂), or a straight or branched alkenylene chain (optionally substituted by hydroxy, mercapto, alkylthio, aryl, cycloalkyl, —N(R⁶)₂, —C(O)OR⁶, or —C(O)N(R⁶)₂);
each R³ is independently alkyl, alkenyl, aryl, aralkyl or aralkenyl; and
R⁹ is —R⁷N(R⁶)C(O)OR⁸, haloalkyl, alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), or N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)p(O)R⁶).

15. The method of claim 14 wherein:
R¹ is hydrogen;
R² is —P(O)(OR⁵)—R⁷—N(R⁵)—S(O)₂—R⁹;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸; —R⁷—N(R⁶)—C(O)OR⁸, and —N(R⁵)—C(NR⁵)—N(R⁵)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain (optionally substituted by hydroxy, mercapto, alkylthio, aryl, cycloalkyl, —N(R⁶)₂, —C(O)OR₂, or —C(O)N(R⁶)₂),
each R⁸ is independently alkyl, alkenyl, aryl, aralkyl or aralkenyl; and
R⁹ is —R⁷N(R⁶)C(O)OR⁸, haloalkyl, alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁵)₂, —C(O)OR⁶, —C(O)N(R⁶)₂ or —N(R⁶)C(O)R⁶), aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), or N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

16. The method of claim 15 wherein:
R¹ is hydrogen;
R² is —P(O)(OR⁵)—R⁷—N(R⁵)—S(O)₂—R⁹;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸; —R⁷—N(R⁶)—C(O)OR⁸, and —N(R⁵)—C(NR⁵)—N(R⁵)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain optionally substituted by hydroxy, mercapto, alkylthio, aryl, cycloalkyl, —N(R⁶)₂, —C(O)OR⁶, or—C(O)N(R⁶)₂,
each R⁸ is independently alkyl, alkenyl, aryl, aralkyl or aralkenyl; and
R⁹ is alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), or aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

17. The method of claim 16 wherein the compound of formula (I) is selected from the group consisting of:
2-(3-(amino)methylphenyl)-3-((1-(3-phenylpropylsulfonyl)amino-2-methylpropyl)-(hydroxy)phosphinoyl)propanoic acid, methyl ester;
2-(3-(t-butoxycarbonylamino)methylphenyl)-3-((1-(3-phenylpropylsulfonyl)amino-2-methylpropyl)(hydroxy)phosphinoyl)propanoic acid;
2-(3-(t-butoxycarbonylamino)methylphenyl)-3-((1-(3-phenylpropylsulfonyl)amino-2-methylpropyl)(hydroxy)phosphinoyl)propanoic acid, methyl ester;
2-(3-(amino)methylphenyl)-3-((1-(3-phenylpropylsulfonyl)amino-2-methylpropyl)-(hydroxy)phosphinoyl)propanoic acid;
(2R)-2-(3-(amino)methylphenyl)-3-(((1R)-1-(3-phenylpropylsulfonyl)amino-2-methylpropyl)(hydroxy)phosphinoyl)propanoic acid;
(2S)-2-(3-(amino)methylphenyl)-3-(((1R)-1-(3-phenylpropylsulfonyl)amino-2-methylpropyl)(hydroxy)phosphinoyl)propanoic acid;
(2R/S)-2-(3-(amino)methylphenyl)-3-(((1S)-1-(3-phenylpropylsulfonyl)amino-2-methylpropyl)(hydroxy)phosphinoyl)propanoic acid;
(2R/S)-2-(3-(amino)methylphenyl)-3-(((1R)-1-(3-phenylpropylsulfonyl)amino-2-methylpropyl)(hydroxy)phosphinoyl)propanoic acid;
(2R)-2-(3-(amino)methylphenyl)-3-(((1S)-1-(3-phenylpropylsulfonyl)amino-2-methylpropyl)(hydroxy)phosphinoyl)propanoic acid;
(2S)-2-(3-(amino)methylphenyl)-3-(((1S)-1-(3-phenylpropyIsulfonyl)amino-2-methylpropyl)(hydroxy)phosphinoyl)propanoic acid;
2-(3-(t-butoxycarbonylamino)methylphenyl)-3-((1-(3-phenylpropylsulfonyl)amino-2-methylpropyl)(ethoxy)phosphinoyl)propanoic acid, t-butyl ester;
2-(3-(amino)methylphenyl)-3-((1-(2-phenylethylsulfonyl)amino-2-methylpropyl)-(hydroxy)phosphinoyl)propanoic acid;
2-(3-(amino)methylphenyl)-3-((1-(benzylsulfonyl)amino-2-methylpropyl)-(hydroxy)phosphinoyl)propanoic acid;
2-(3-(amino)methylphenyl)-3-((1-(2-(naphth-1-yl)ethylsulfonyl)amino-2-methylpropyl)-(hydroxy)phosphinoyl)propanoic acid;
2-(3-(amino)methylphenyl)-3-((1-(3-(4-methoxyphenyl)propylsulfonyl)amino-2-methylpropyl)(hydroxy)phosphinoyl)propanoic acid;
2-(3-(amino)methylphenyl)-3-((1-(2-(4-methoxyphenyl)ethylsulfonyl)amino-2-methylpropyl)(hydroxy)phosphinoyl)propanoic acid;
2-(3-(amino)methylphenyl)-3-((1-(methylsulfonyl)amino-2-methylpropyl)(hydroxy)phosphinoyl)propanoic acid;
2-(3-(amino)methylphenyl)-3-((1-(2-benzyloxyethylsulfonyl)amino-2-methylpropyl)-(hydroxy)phosphinoyl)propanoic acid;
2-(3-(amino)methylphenyl)-3-((1-(2-hydroxyethylsulfonyl)amino-2-methylpropyl)-(hydroxy)phosphinoyl)propanoic acid;
2-(3-aminophenyl)-3-((1-(3-phenylpropylsulfonyl)amino-2-methylpropyl)-(hydroxy)phosphinoyl)propanoic acid;
2-(3-guanidinophenyl)-3-((1-(3-phenylpropylsulfonyl)amino-2-methylpropyl)(hydroxy)-phosphinoyl)propanoic acid;
2-(3-(amino)methylphenyl)-3-((1-(4-phenylbutylsulfonyl)amino-2-methylpropyl)-(hydroxy)phosphinoyl)propanoic acid, and
2-(3-(amino)methylphenyl)-3-((1-(2-phenylethenylsulfonyl)amino-2-methylpropyl)-(hydroxy)phosphinoyl)propanoic acid.

18. The method of claim 15 wherein:
R¹ is hydrogen;
R² is —P(O)(OR⁵)—R⁷—N(R⁵)—S(O)₂—R⁹;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸; —R⁷—N(R⁶)—C(O)OR⁸, and —N(R⁵)—C(NR⁵)—N(R⁵)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain optionally substituted by hydroxy, mercapto, alkylthio, aryl, cycloalkyl, —N(R⁶)₂, —C(O)OR⁶, or—C(O)N(R⁶)₂,
each R⁸ is independently alkyl, alkenyl, aryl, aralkyl or aralkenyl; and
R⁹ is aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

19. The method of claim 18 wherein the compound of formula (I) is selected from the group consisting of:
2-(3-(amino)methylphenyl)-3-((1-(naphth-1-ylsulfonyl)amino-2-methylpropyl)(hydroxy)-phosphinoyl)propanoic acid;
2-(3-(amino)methylphenyl)-3-((1-(3-trifluoromethylphenylsulfonyl)amino-2-methyl-propyl)(hydroxy)phosphinoyl)propanoic acid;
2-(3-(amino)methylphenyl)-3-((1-(4-pentylphenylsulfonyl)amino-2-methylpropyl)(hydroxy)phosphinoyl)propanoic acid;
2-(3-(amino)methylphenyl)-3-((1-(4-acetamidophenylsulfonyl)amino-2-methylpropyl)(hydroxy)phosphinoyl)propanoic acid;
2-(3-(amino)methylphenyl)-3-((1-(4-phenylphenylsulfonyl)amino-2-methylpropyl)-(hydroxy)phosphinoyl)propanoic acid; and
2-(3-(amino)methylphenyl)-3-((1-(phenylsulfonyl)amino-2-methylpropyl)(hydroxy)-phosphinoyl)propanoic acid.

20. The method of claim 15 wherein:
R¹ is hydrogen;
R² is —P(O)(OR⁵)—R⁷—N(R⁵)—S(O)₂—R⁹;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸; —R⁷—N(R⁶)—C(O)OR⁸, —N(R⁶)—C(O)—R⁶, —R⁷—N(R⁶)—C(O)—R⁶, and —N(R⁵)—C(NR⁵)—N(R⁵)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain (optionally substituted by hydroxy, mercapto, alkylthio, aryl, cycloalkyl, —N(R⁶)₂, —C(O)OR⁶, or —C(O)N(R⁶)₂),
each R⁵ is independently alkyl, alkenyl, aryl, aralkyl or aralkenyl; and
R⁹ is —R⁷—N(R⁶)—C(O)OR⁸.

21. The method of claim 20 wherein the compound of formula (I) is
2-(3-(amino)methylphenyl)-3-((1-(3-phenyl-2-(benzyloxycarbonyl)aminopropylsulfonyl)-amino-2-methylpropyl)(hydroxy)phosphinoyl)propanoic acid.

22. The method of claim 15 wherein:
R¹ is hydrogen;
R²is —P(O)(OR⁵)—R⁷—N(R⁵)—S(O)₂—R⁹;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸; —R⁷—N(R⁶)—C(O)OR⁸, —N(R⁶)—C(O)—R⁶, —R⁷—N(R⁶)—C(O)—R⁶, and —N(R⁵)—C(NR⁵)—N(R⁵)₂;
each R⁵ is independently hydrogen, alkyl or aralkyl;
each R is independently hydrogen, alkyl, alkenyl, alkynyl, aryl, aralkyl or aralkenyl;
each R⁷ is independently a straight or branched alkylene chain (optionally substituted by hydroxy, mercapto, alkylthio, aryl, cycloalkyl, —N(R⁶)₂, —C(O)OR⁶, or—C(O)N(R⁶)₂),
each R⁸ is independently alkyl, alkenyl, aryl, aralkyl or aralkenyl; and
R⁹ is N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

23. The method of claim 22 wherein the compound of formula (I) is selected from the group consisting of:
2-(3-(amino)methylphenyl)-3-((1-(thien-2-ylsulfonyl)amino-2-methylpropyl)-(hydroxy)phosphinoyl)propanoic acid; and
2-(3-(amino)methylphenyl)-3-((1-(benzothiadiazolylsulfonyl)amino-2-methylpropyl)-(hydroxy)phosphinoyl)propanoic acid.

24. The method of claim 1 wherein:
R¹ is hydrogen;
R² is —P(O)(OR⁵)—R⁷—N(R⁵)—C(O)OR⁸;
R³ is —C(O)OR⁶;
R⁴ is unsubstituted phenyl or unsubstituted N-heterocyclyl;
each R⁵ is independently hydrogen, alkyl or aralkyl;
each R⁶ is independently hydrogen, alkyl, alkenyl, alkynyl, aryl, aralkyl or aralkenyl;
each R⁷ is a straight or branched alkylene chain optionally substituted by aryl, —N(R⁶)₂ or —C(O)OR⁶; and
R⁸ is alkyl, alkenyl, aryl, aralkyl or aralkenyl.

25. The method of claim 24 wherein the compound of formula (I) is selected from the group consisting of:
2-phenyl-3-((1-(benzyloxycarbonyl)amino-2-methylpropyl)(hydroxy)phosphinoyl)-propanoic acid; and
2-tetrahydroisoquinolinyl-3-((1-(benzyloxycarbonyl)amino-2-methylpropyl)-(hydroxy)phosphinoyl)propanoic acid.

26. The method of claim 1 wherein the administration of the compound to a patient is prior to PCI.

27. The method of claim 1 wherein the compound is combined with other therapeutic agents for administration or the compound is coadministered with other therapeutic agents.

28. A method of enhancing myocardial reperfusion and facilitating PCI comprising administering to a patient in need thereof a therapeutically effective amount of a compound of formula (II) or a pharmaceutically acceptable salt thereof: 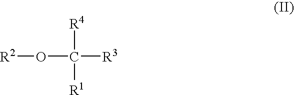
wherein:
R¹ is hydrogen, alkyl, alkenyl, aryl or aralkenyl;
R² is —P(O)(OR⁵)₂, —P(O)(O R⁵)R⁶, —P(O)(OR⁵)—R⁷—N(R⁶)₂, —P(O)(OR⁵)—R⁷—C(O)—R⁸, —P(O)(OR⁵)—R⁷—N(R⁵)—C(O)OR⁸, —P(O)(OR⁵)—R⁷—N(R⁵)—C(O)—R⁷—N(R⁵)—C(O)OR⁸, —P(O)(OR⁵)—R⁷—N(R⁵)—S(O)₂—R⁹, or —P(O)(OR⁵)—R⁷—N(R⁵)—C(S)—N(R⁶)₂;
R³ is tetrazole, —C(O)OR⁶, —C(O)O—R⁷—OC(O)R⁵, —S(O)OR⁵, —S(O)₂OR⁵, —P(O)(OR⁵)₂, —P(O)(OR⁵)R⁶, or —B(OR⁵)₂;
R⁴ is aryl optionally substituted by one or more substituents selected from the group consisting of alkyl, halo, haloalkyl, haloalkoxy, mercapto, alkylthio, phenyl, cycloalkyl, nitro, cyano, —OR⁶, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R^{—N(R}⁵)—C(O)—N(R⁶)₂ or —N(R⁵)—C(O)—R⁷—N(R⁶²), or wherein a nitrogen atom in the N-heterocyclyl may be optionally substituted by —C(NR⁵)—N(R⁵)₂, —C(NR⁵)—R⁵, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷is independently cycloalkylene (optionally substituted by alkyl), a straight or branched alkylene chain (optionally substituted by hydroxy, mercapto, alkylthio, aryl, cycloalkyl, —N(R⁶)₂, —C(O)OR⁶, or —C(O)N(R⁶)₂), or a straight or branched alkenylene chain (optionally substituted by hydroxy, mercapto, alkylthio, aryl, cycloalkyl, —N(R⁶)₂, —C(O)OR⁶, or —C(O)N(R⁶)₂);
each R³ is independently alkyl, alkenyl, aryl, aralkyl or aralkenyl; and
R⁹ is —R⁷N(R⁶)C(O)OR⁸, haloalkyl, alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), or N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶);
provided that when R³ is —C(O)OH or when R⁴ is a substituted aryl or substituted N-heterocyclyl,
R² can not be —P(O)(OR⁵)—R⁷—N(H)—C(O)OR⁸ or
—P(O)(OR⁵)—R⁷ —N(H)—C(O)—R⁷—N(R⁵)—C(O)OR⁸;
as a single stereoisomer, a mixture of stereoisomers, or as a racemic mixture of stereoisomers.

29. The method of claim 28 wherein:
R¹ is hydrogen;
R²is —P(O)(OR⁵)R⁶, —P(O)(O R⁵)—R⁷—N(R⁶)₂, or —P(O)(OR⁵)—R⁷—N(R⁵)—C(S)—N(R⁶)₂;
R³is tetrazole, —C(O)OR⁶ or —C(O)O—R⁷—OC(O)R⁵;
R⁴is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷ —N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
each R⁵ is independently hydrogen, alkyl or aralkyl;
each R⁶ is independently hydrogen, alkyl, alkenyl, alkynyl, aryl, aralkyl or aralkenyl;
each R⁷is a straight or branched alkylene chain (optionally substituted by hydroxy, mercapto, alkylthio, aryl, cycloalkyl, —N(R⁶)₂, —C(O)OR⁶, or —C(O)N(R⁶)₂); and
each R⁸ is independently alkyl, alkenyl, aryl, aralkyl or aralkenyl.

30. The method of claim 29 wherein the compound of formula (II) is selected from the group consisting of:
2-(3-guanidinophenyl)-2-((1-(2-phenylethyl)amino-2-methylpropyl)-(hydroxy)phosphinoyloxy)ethanoic acid;
2-(3-aminophenyl)-2-((phenyl)(hydroxy)phosphinoyloxy)ethanoic acid;
2-(3-guanidinophenyl)-2-((1-amino-2-methylpropyl)(hydroxy)phosphinoyloxy)ethanoic acid; and
2-(3-guanidinophenyl)-2-((1-(benzylaminothiocarbonyl)amino-2-methylpropyl)-(hydroxy)phosphinoyloxy)ethanoic acid.

31. The method of claim 28 wherein:
R¹ is hydrogen;
R² is —P(O)(OR⁵)—R⁷—N(R⁵)—S(O)₂—R⁹;
R³is tetrazole, —C(O)OR⁶ or —C(O)O—R⁷—OC(O)R⁵;
R⁴is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
each R⁵ is independently hydrogen, alkyl or aralkyl;
each R⁶ is independently hydrogen, alkyl, alkenyl, alkynyl, aryl, aralkyl or aralkenyl;
each R⁷is a straight or branched alkylene chain (optionally substituted by hydroxy, mercapto, alkylthio, aryl, cycloalkyl, —N(R⁶)₂, —C(O)OR⁶, or —C(O)N(R⁶)₂);
each R⁸ is independently alkyl, alkenyl, aryl, aralkyl or aralkenyl; and
R⁹ is alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), or aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

32. The method of claim 31 wherein the compound of formula (II) is selected from the group consisting of:
2-(3-guanidinophenyl)-2-((1-(benzylsulfonyl)amino-2-methylpropyl)(hydroxy)-phosphinoyloxy)ethanoic acid; and
2-(3-guanidinophenyl)-2-((1-(2-phenylethenylsulfonyl)amino-2-methylpropyl)-(hydroxy)phosphinoyloxy)ethanoic acid.

33. The method of claim 28 wherein:
R¹ is hydrogen;
R² is —P(O)(OR⁵)₂, —P(O)(OR⁵)R⁶, —P(O)(OR⁵)—R⁷—N(R⁶)₂, —P(O)(OR⁵)—R⁷—C(O)—R⁸, —P(O)(OR⁵)—R⁷ —N(R⁵)—C(O)OR⁸, —P(O)(OR⁵)—R⁷—N(R⁵)—C(O)—R⁷—N(R⁵)—C(O)OR¹¹, —P(O)(OR⁵)—R⁷—N(R⁵)—S(O)₂—R⁹, or —P(O)(OR⁵)—R⁷—N(R⁵)—C(S)—N(R⁶)₂;
R³is tetrazole;
R⁴is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷ —N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶²), or wherein a nitrogen atom in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵is independently hydrogen, alkyl or aralkyl;
each R is independently hydrogen, alkyl, alkenyl, alkynyl, aryl, aralkyl or aralkenyl;
each R⁷is independently cycloalkylene (optionally substituted by alkyl), a straight or branched alkylene chain (optionally substituted by hydroxy, mercapto, alkylthio, aryl, cycloalkyl, —N(R⁶)₂, —C(O)OR⁶, or —C(O)N(R⁶)₂), or a straight or branched alkenylene chain (optionally substituted by hydroxy, mercapto, alkylthio, aryl, cycloalkyl, —N(R⁶)₂, —C(O)OR⁶, or —C(O)N(R⁶)₂);
each R⁸ is independently alkyl, alkenyl, aryl, aralkyl or aralkenyl; and
R⁹ is —R⁷N(R⁶)C(O)OR⁸, haloalkyl, alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), or N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

34. The method of claim 33 wherein the compound of formula (II) is
2-methyl-1-[1-(3-guanidinophenyl)-1-tetrazolylmethoxy](hydroxy)phosphinoyl-propylcarbamic acid, benzyl ester.

35. The method of claim 28 wherein the administration of the compound to a patient is prior to PCI.

36. The method of claim 8 wherein the compound is combined with other therapeutic agents for administration or the compound is co-administered with other therapeutic agents.

37. A method of enhancing myocardial reperfusion and facilitating PCI comprising administering to a patient in need thereof a therapeutically effective amount of a compound of formula (III), or a pharmaceutically acceptable salt thereof: 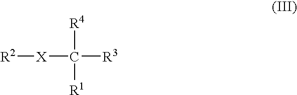
wherein:
X is —CH₂— or —O—;
R¹ is hydrogen, alkyl, alkenyl, aryl or aralkenyl;
R² is —P(O)(OR⁵)—R⁷—N(R⁵)—C(O)R⁶, —P(O)(OR⁵)—R⁷—N(R⁵)—C(O)OR⁸ or —P(O)(OR⁵)—R⁷—N(R⁵)—C(O)—R⁷—N(R⁵)—C(O)OR⁸,
R³is —C(O)OH;
R⁴is aryl optionally substituted by one or more substituents selected from the group consisting of alkyl, halo, haloalkyl, haloalkoxy, mercapto, alkylthio, phenyl, cycloalkyl, nitro, cyano, —OR⁶, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the
N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently cycloalkylene (optionally substituted by alkyl), a straight or branched alkylene chain (optionally substituted by hydroxy, mercapto, alkylthio, aryl, cycloalkyl, —N(R⁶)₂, —C(O)OR⁶, or —C(O)N(R⁶)₂), or a straight or branched alkenylene chain (optionally substituted by hydroxy, mercapto, alkylthio, aryl, cycloalkyl, —N(R⁶)₂, —C(O)OR⁶, or —C(O)N(R⁶)₂); and
each R⁸ is independently alkyl, alkenyl, aryl, aralkyl or aralkenyl;
as a single stereoisomer, a mixture of stereoisomers, or as a racemic mixture of stereoisomers.

38. The method of claim 37 wherein:
X is —O—;
R² is —P(O)(OR⁵)—R⁷—N(R⁵)—C(O)OR⁸; and
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—N(R⁶)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂).

39. The method of claim 38 wherein the compound of formula (III) is selected from the group consisting of:
2-(3-guanidinophenyl)-2-((1-(benzyloxycarbonyl)aminoethyl)(hydroxy)-phosphinoyloxy)ethanoic acid;
2-(3-guanidinophenyl)-2-(((benzyloxycarbonyl)aminomethyl)(hydroxy)-phosphinoyloxy)ethanoic acid;
2-(3-guanidinophenyl)-2-((1-(benzyloxycarbonyl)amino-2-methylpropyl)(hydroxy)phosphinoyloxy)ethanoic acid;
2-(3-guanidinophenyl)-2-((1-(benzyloxycarbonyl)aminohexyl)-(hydroxy)phosphinoyloxy)ethanoic acid;
2-(3-aminophenyl)-2-((1-(benzyloxycarbonyl)amino-2-methylpropyl)(hydroxy)phosphinoyloxy)ethanoic acid;
2-(3-guanidinophenyl)-2-((1-(benzyloxycarbonyl)amino-3-methylbutyl)(hydroxy)phosphinoyloxy)ethanoic acid;
2-(2-chloro-3-guanidinophenyl)-2-((1-(benzyloxycarbonyl)amino-2-methylpropyl)(hydroxy)phosphinoyloxy)ethanoic acid;
2-(3-guanidinophenyl)-2-((1-(benzyloxycarbonyl)amino-1-phenylmethyl)(hydroxy)phosphinoyloxy)ethanoic acid;
2-(2-fluoro-3-guanidinophenyl)-2-((1-(benzyloxycarbonyl)amino-2-methylpropyl)(hydroxy)phosphinoyloxy)ethanoic acid;
2-(3-guanidinophenyl)-2-((1-(benzyloxycarbonyl)amino-1-cyclohexylmethyl)(hydroxy)phosphinoyloxy)ethanoic acid;
2-(2-methyl-3-guanidinophenyl)-2-((1-(benzyloxycarbonyl)amino-2-methylpropyl)(hydroxy)phosphinoyloxy)ethanoic acid;
2-(3-(amino)methylphenyl)-2-((1-(benzyloxycarbonyl)amino-2-methylpropyl)(hydroxy)phosphinoyloxy)ethanoic acid;
2-(3-(guanidinomethyl)phenyl)-2-((1-(benzyloxycarbonyl)amino-2-methylpropyl)(hydroxy)phosphinoyloxy)ethanoic acid;
2-(3-(1-iminoethylaminophenyl))-2-((1-(benzyloxycarbonyl)amino-2-methylpropyl)(hydroxy)phosphinoyloxy)ethanoic acid;
2-(3-(t-butoxycarbonylamino)methylphenyl)-2-((1-(benzyloxycarbonyl)amino-2-methylpropyl)(hydroxy)phosphinoyloxy)ethanoic acid;
2-(3-(ethoxycarbonylamino)methylphenyl)-2-((1-(benzyloxycarbonyl)amino-2-methylpropyl)(hydroxy)phosphinoyloxy)ethanoic acid;
2-(3-(isopropoxycarbonylamino)methylphenyl)-2-((1-(benzyloxycarbonyl)amino-2-methylpropyl)(hydroxy)phosphinoyloxy)ethanoic acid;
2-(3-(2,2-dimethylpropylcarbonylamino)methylphenyl)-2-((1-(benzyloxycarbonyl)amino-2-methylpropyl)(hydroxy)phosphinoyloxy)ethanoic acid;
2-(3-guanidinophenyl)-2-((1-(2-phenylethylcarbonyl)amino-2-methylpropyl)(hydroxy)phosphinoyloxy)ethanoic acid; and
2-(3-guanidinophenyl)-2-((1-(2-phenylethenylcarbonyl)amino-2-methylpropyl)(hydroxy)phosphinoyloxy)ethanoic acid.

40. The method of claim 37 wherein:
X is —O—;
R² is —P(O)(OR⁵)—R⁷—N(R⁵)—C(O)—R⁷—N(R⁵)—C(O)OR⁸; and
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—N(R⁶)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂).

41. The method of claim 40 wherein the compound of formula (III) is selected from
2-(3-guanidinophenyl)-2-[(1-(1-benzyloxycarbonylamino-2-(4-hydroxyphenyl)-ethylcarbonyl)amino-2-methylpropyl)(hydroxy)phosphinoyloxy]ethanoic acid;
2-(3-guanidinophenyl)-2-[(1-(1-benzyloxycarbonylamino-2-phenylethylcarbonyl)amino-2-methylpropyl)(hydroxy)phosphinoyloxy]ethanoic acid;
2-(2-fluoro-3-guanidinophenyl)-2-[(1-(1-benzyloxycarbonylamino-2-phenylethylcarbonyl)amino-2-methylpropyl)(hydroxy)phosphinoyloxy]ethanoic acid;
2-(3-guanidinophenyl)-2-[(1-(1-phenylcarbonylamino-2-phenylethylcarbonyl)amino-2-methylpropyl)(hydroxy)phosphinoyloxy]ethanoic acid;
2-(3-guanidinophenyl)-2-[(1-(1-ethoxycarbonylamino-2-phenylethylcarbonyl)amino-2-methylpropyl)(hydroxy)phosphinoyloxy]ethanoic acid;
2-(3-guanidinophenyl)-2-[(1-(1-benzyloxycarbonylamino-3-phenylpropylcarbonyl)amino-2-methylpropyl)(hydroxy)phosphinoyloxy]ethanoic acid; and
2-(3-(amino)methylphenyl)-2-[(1-(1-benzyloxycarbonylamino-3-phenylpropylcarbonyl)amino-2-methylpropyl)(hydroxy)phosphinoyloxy]ethanoic acid.

42. The method of claim 37 wherein:
X is —CH₂—; R² is —P(O)(OR⁵)—R⁷—N(R⁵)—C(O)R⁶ or —P(O)(OR⁵)—R⁷—N(R⁵)—C(O)OR⁸; and
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—N(R⁶)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂).

43. The method of claim 42 wherein the compound of formula (III) is selected from the group consisting of:
2-(3-(amino)methylphenyl)-3-((1-(methylcarbonyl)amino-2-methylpropyl)(hydroxy)-phosphinoyl)propanoic acid;
2-(3-(hydrazinocarbonyl)phenyl)-3-((1-(benzyloxycarbonyl)amino-2-methylpropyl)-(hydroxy)phosphinoyl)propanoic acid;
2-(3-guanidinophenyl)-3-((1-(benzyloxycarbonyl)aminoethyl)(hydroxy)phosphinoyl)-propanoic acid;
2-(3-guanidinophenyl)-3-((1-(benzyloxycarbonyl)amino-3-methylbutyl)(hydroxy)phosphinoyl)propanoic acid;
2-(3-guanidinophenyl)-3-(((benzyloxycarbonyl)aminomethyl)(hydroxy)phbsphinoyl)-propanoic acid;
2-(3-guanidinophenyl)-3-((1-(benzyloxycarbonyl)amino-2-methylpropyl)(hydroxy)-phosphinoyl)propanoic acid;
2-(2-chloro-5-guanidinophenyl)-3-((1-(benzyloxycarbonyl)amino-2-methylpropyl)(hydroxy)phosphinoyl)propanoic acid;
2-(3-(amino)methylphenyl)-3-((1-(benzyloxycarbonyl)amino-2-methylpropyl)(hydroxy)-phosphinoyl)propanoic acid; and
2-(3-(amino)methylphenyl)-3-((1-(2-phenylethylcarbonyl)amino-2-methylpropyl)-(hydroxy)phosphinoyl)propanoic acid.

44. The method of claim 37 wherein:
X is —CH₂—;
R² is —P(O)(OR⁵)—R⁷—N(R⁵)—C(O)—R⁷—N(R⁵)—C(O)OR⁸; and
R⁴ is aryl optionally substituted by one or more substituents selected from the group consisting of alkyl, halo, nitro, cyano, —N(R⁶)₂, —R⁷ —N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—N(R⁶)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂).

45. The method of claim 44 wherein the compound of formula (III) is selected from the group consisting of:
2-(3-guanidinophenyl)-3-(((1-benzyloxycarbonylamino-2-phenylethyl)carbonylaminomethyl)(hydroxy)phosphinoyl)propanoic acid; and
2-(3-guanidinophenyl)-3-(((1-benzyloxycarbonylamino-2-phenylethyl)carbonylaminomethyl)(hydroxy)phosphinoyl)propanoic acid.

46. The method of claim 37 wherein the administration of the compound to a patient is prior to PCI.

47. The method of claim 37 wherein the compound is combined with other therapeutic agents for administration or the compound is coadministered with other therapeutic agents.

48. A method of enhancing myocardial reperfusion and facilitating PCI comprising administering to a patient in need thereof a therapeutically effective amount of a TAFI inhibitor.

49. The method of claim 48 wherein the administration of the compound to a patient is prior to PCI.

50. The method of claim 49 wherein the compound is combined with other therapeutic agents for administration or the compound is coadministered with other therapeutic agents.
